(19) United States
(12) Patent Application Publication
(10) Pub. No.: US 2024/0327475 A1
(43) Pub. Date: Oct. 3, 2024

(54) ICAM-1 TARGETED FUSION ENZYMES

(71) Applicant: University of Maryland, College Park, College Park, MD (US)

(72) Inventors: Silvia Muro, Gaithersburg, MD (US); Jing Chen, Nanjing (CN); Melani Solomon, Laurel, MD (US); Kevin Gray, Washington, DC (US)

(21) Appl. No.: 18/416,843

(22) Filed: Jan. 18, 2024

Related U.S. Application Data
(63) Continuation of application No. 17/571,415, filed on Jan. 7, 2022, now Pat. No. 11,912,745.
Application No. 17/571,415 is a continuation of application No. 16/951,774, filed on Nov. 18, 2020, now Pat. No. 11,248,029.
(60) Provisional application No. 62/936,988, filed on Nov. 18, 2019.

(51) Int. Cl. C07K 14/47; C12N 9/16; C12N 9/24; C12N 9/40

(52) U.S. Cl. CPC C07K 14/47; C12N 9/16; C12N 9/2402; C12N 9/2465; C12Y 302/01022; C12Y 302/01045; C07K 2319/055; C07K 2319/21; C07K 2319/33; C07K 2319/50

(57) ABSTRACT

Proteins, nucleic acids encoding the proteins, compositions comprising the proteins, and methods are provided. The proteins have the ability to be self-targeted to ICAM-1 and, if desired, enzymatically-released at acidic pH. The ICAM-1-targeting peptides are provided as single copies or multiples repeats, and can be separated by linkers from the enzyme segment, from which the ICAM-1 targeting peptides can be released, if desired, at acidic pH. These fusion proteins enhance the activity of the enzyme segment within or liberated from the fusion protein, and provide increased recognition and targeting of diseased organs, transport from the bloodstream across the endothelium into said diseased organ, and intracellular uptake and lysosomal trafficking by cells in them, both in peripheral tissues and the central nervous system. Representative nucleotide and amino acid sequences of these fusion proteins, as well as in vitro, cellular, and in vivo animal data are provided. The described proteins can be used as a protein therapy, a gene therapy, or an implanted cell therapy.

### CROSS-REFERENCE TO RELATED APPLICATIONS

This application is a continuation of U.S. patent application Ser. No. 17/571,415, filed Jan. 7, 2022, which is a continuation of U.S. patent application Ser. No. 16/951,774, filed Nov. 18, 2020, now U.S. Pat. No. 11,248,029, which claims priority to U.S. provisional patent application No. 62/936,988, filed Nov. 18, 2019, the entire disclosures of each of which are incorporated herein by reference.

### SEQUENCE LISTING

The instant application contains a Sequence Listing which is submitted in .xml format and is hereby incorporated by reference in its entirety. Said .xml file is named “070919_00143_ST26.xml”, was created on Jan. 18, 2024, and is 81,051 bytes in size.

### FIELD

The present disclosure relates generally to compositions and methods for treating lysosomal storage diseases (LSDs) and other diseases where lysosomal enzyme activities are beneficial.

### BACKGROUND

LSDs are caused by defects in one or more hydrolytic enzymes of lysosomes in cells that digest biomacromolecules for cellular housekeeping. Lack of this function results in unwanted build-up of these molecules in cells and depending on the enzyme affected, specific substrate processing is impaired, and severity ranges from life-long debilitation to death. Supplementing defective enzymes by enzyme replacement therapy (ERT) is the most accepted treatment and a “universal” approach at present. However, there is an ongoing need for compositions and methods for use as ERT treatments. In addition, the activity of these lysosomal enzymes is also applicable to the treatment of other maladies. For instance, ceramide, the product of the activity of acid sphingomyelinase which is deficient in the LSD called types A and B Niemann-Pick disease, can induce cellular apoptosis when in excess. Hence, ERT methods and compositions for treatment of types A and B Niemann-Pick disease can also be used for cancer treatment. Similarly, mutations and defects in lysosomal enzyme glucocerebrosidase, which is deficient in the LSD called Gaucher disease, constitute a main hallmark in Parkinson's disease. It has been shown that increased activity of this enzyme improves the outcome of Parkinson's in animal models. Hence, ERT methods and compositions for treatment Gaucher disease can also be used for treatment of Parkinson's, but to date there remains an ongoing need for improved compositions and methods for prophylaxis and/or treatment of such ERT conditions. The present disclosure is pertinent to these needs.

### BRIEF SUMMARY

The present disclosure provides compositions and methods that are useful for treating a variety of LSDs. The compositions include fusion proteins, for use in treating one or more LSDs, or additional diseases which may benefit from these enzyme activities.

Data presented in this disclosure demonstrate that, unexpectedly and unpredictably, the described fusion proteins exhibit enhanced enzymatic activity in conditions mimicking lysosomes, such as lysosomal pH, both as such fusion proteins and also after the enzyme segment has been liberated from the fusion protein, relative to the same enzyme that is not provided in a fusion protein context. This enhanced activity cannot be explained solely by the precise enzyme segment sequence used to form the fusion protein, because when the same enzyme segment is used to produce an enzyme without fusion to ICAM-1 targeting peptides, its activity is lower than that of the fusion protein or the enzymatic segment liberated from the fusion protein.

Data presented in this disclosure also support the use of the described fusion proteins for improved effects in cellular models and in mouse organs, including but not necessarily limited to the lung and brain, the latter of which no previously described enyme replacement therapy has been able to access in a therapeutic dose. With respect to the fusion proteins provided by the disclosure, they generally comprise: i) one or more intercellular adhesion molecule-1 (ICAM-1) targeting segments; ii) an enzyme segment that is catalytically active at the pH of a lysosome; iii) optionally a first protease cleavage sequence segment between i) and ii), and optionally, one or more of: iv) a secretion signal; v) a protein purification tag; and vi) a second protease cleavage signal, such as for use in protein purification of iv) and v) from the final product. The form and content of the fusion protein can be changed depending on, for example, its method of delivery.

In embodiments, the ICAM-1 targeting segment comprises amino acid SEQ ID NO 1 NNQKIVNIKEKVAQIEA (2γ3) or respective nucleotide sequence, which are comprised in fusion proteins containing amino acid or nucleotide SEQ ID NO 2, 3, 4, 5, 7, 8, 10, 11, 13, 14, 15, 16, 18, 19, 21, 22. This is in contrast to non-targeted enzyme sequences shown as control in some embodiments, such as amino acid or nucleotide SEQ ID NO 6, 9, 12, 17, 20, and 23. In both cases, that of fusion proteins or control non-targeted enzymes, nucleotide sequences provided include codon optimization for expression in mammalian cells, but this should not limit the use of other codon sequences encoding similar amino acids to those described. In some embodiments, the ICAM-1 targeting sequence may be repeated, and thus may appear in the fusion protein more than once. In embodiments, the enzyme segment comprises at least one of Acid sphingomyelinase (ASM), Alpha galactosidase (αGal), or Glucocerebrosidase (GCase), or a catalytically active fragment of any of said enzymes. The fusion protein may be delivered to an individual in need thereof using any of a variety of delivery forms and methods. The disclosure includes administration of the described fusion proteins, and polynucleotides encoding them, such as RNA or DNA or a suitable expression vector, or cells producing said proteins or nucleic acids or vectors. Thus, expression vectors, mRNA, and cDNAs encoding the fusion proteins are included. Also included are cells, including but not limited to mammalian cells, which further include but are not limited to human cells, that produce described fusion proteins or comprise the described vectors containing said sequences, as well as said cells to which a described fusion protein has bound, and/or which have internalized the fusion protein. The described proteins can be used as a protein therapy, a gene therapy, or by way of an implanted cell therapy, wherein the implanted cells express a described fusion protein. Organelles and cellular vesicles, such as lysosomes and exosomes, which comprise an intact or cleaved fusion protein, and organs, such as the lungs, liver or brain, to which these fusion proteins have been delivered are also included within the scope of the disclosure.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Schematics of representative fusion proteins. (A.) Human acid sphingomyelinase (ASM) with one copy of the 2γ3 ICAM-1-targeting peptide at the amino terminus; (B.) human ASM with five tandem-repeats of the 2γ3 ICAM-1-targeting peptide at the amino terminus; (C.) human ASM with ten tandem-repeats of the 2γ3 ICAM-1-targeting peptide at the amino terminus; (D.) human ASM with five tandem-repeats of the 2γ3 ICAM-1-targeting peptide at the carboxyl terminus; (E.) human ASM control; (F.) human alpha galactosidase (αGal) with one copy of the 2γ3 ICAM-1-targeting peptide at the amino terminus; (G.) human αGal with five tandem-repeats of the 2γ3 ICAM-1-targeting peptide at the carboxyl terminus; (H.) human αGal control; (I.) human glucocerebrosidase (GCase) with one copy of the 2γ3 ICAM-1-targeting peptide at the amino terminus; (J.) human GCase with five tandem-repeats of the 2γ3 ICAM-1-targeting peptide at the amino terminus; and (K.) human GCase control.

FIG. 2. Cartoon depictions of in silico simulations of the structure of fusion protein from FIG. 1: (A) ASM fusion A in FIG. 1, (B) ASM fusion B in FIG. 1, (C) ASM fusion C in FIG. 1, (D) ASM fusion D in FIG. 1, (E) αGal fusion G in FIG. 1, and (F) GCase fusion J in FIG. 1.

FIG. 3. Photographs of gels after protein electrophoresis followed by western blotting of fusion proteins secreted by CHO cells. (A) prior to and (B) after fusion protein affinity purification via His-tag domain.

FIG. 4. Photographs of gels after protein electrophoresis followed by Coomassie blue after purification of (A) the same fusion enzyme from different cell sources or (B) different fusion enzymes in the same cell source. The data show reproducibility and purity of ICAM-1-targeted fusion enzymes.

FIG. 5. Photographs of gels after protein electrophoresis followed by Western blotting of ICAM-1-targeted fusion enzymes. (A) Prior to and after cleavage with enterokinase. (B) Prior to and after cleavage with cathepsin.

FIG. 6. Table (A.) showing comparative in vitro enzymatic activity of fusion proteins and respective non-fusion enzyme control and enterokinase (EK) cleaved fusion proteins, prior and after release with cathepsin B, at lysosomal versus neutral pH, and for fusions produced in different cell lines. (B., C., D.) Enzymatic activity, under lysosomal conditions of fusion proteins ((B.), fusion ASM; (C.) fusion GCase; (D.) fusion α-Gal) compared to respective control non-targeted enzymes.

FIG. 7. Graph showing cell binding and internalization of an ICAM-1-targeted fusion enzyme compared to respective control non-targeted enzyme.

FIG. 8. Graph (A.) and fluorescence microscopy images (B.) showing, respectively, uptake and lysosomal trafficking of an ICAM-1-targeted fusion enzyme by induced pluripotent stem cells (iPS)-derived Gaucher disease neurons, compared to respective control non-targeted enzyme.

FIG. 9. Results showing reduction of lysosomal storage in patient cells (fibroblasts) by ICAM-1-targeted fusion enzymes. (A) Fluorescence microscopy images, and (B) graph showing quantification of the level of a fluorescent substrate analogue degraded by fusion protein or non-fusion control delivered to cells from a type A Niemann-Pick patient. (C), (D) Graphs showing quantifications similar to (B) in the case of fusion proteins delivered to cells from Gaucher disease and Fabry disease, respectively. In all cases, graphs show increased therapeutic degradation of the respective substrate analog by the fusion protein compared to non-targeted control.

FIG. 10. Fluorescent micrographs (top) and quantification graph (bottom) showing that ICAM-1-targeted fusion enzymes normalize the size of lysosomes in Gaucher disease iPS-neurons, which are aberrantly enlarged compared to wildtype cells. Commercial Cerezyme only slightly attenuates the said lysosomal engorgement.

FIG. 11. Fluorescent micrographs showing lack of cytotoxicity of ICAM-1-targeted fusion proteins. (A) iPS-derived wildtype neurons compared to Gaucher disease neurons. (B) Gaucher neurons treated with control hydrogen peroxide, which is known to be cytotoxic, or with fusion protein.

FIG. 12. Model and graphical results showing transcytosis of ICAM-1-targeted fusion enzymes across models of the blood-brain barrier (BBB) and uptake by subjacent neurons. (A) Multi-cellular Transwell model. (B) Graph showing the concentration of fusion protein or control non-targeted enzyme in the apical or basolateral transwell chambers, demonstrating barrier function. (C) Graph showing that ICAM-1 blockage reduces the amount of fusion protein that interacted with the BBB. (D) Graph showing much higher BBB entrapment of the non-targeted enzyme compared to respective fusion protein. (E) Graph showing much higher uptake of fusion protein by Gaucher disease neurons compared to non-targeted enzyme.

FIG. 13. Graphs showing circulation of ICAM-1-targeted fusion enzymes in mice. (A) A fusion protein produced in two different cell sources compared to a non-targeted enzyme from which Olipudase® is derived. (B) Circulation of the same fusion protein from two different cell sources, before and after cleavage (Cl) of the His-tag domain.

FIG. 14. Graphs showing enhanced (A) lung and (B) brain distribution of ICAM-1-targeted fusion enzymes in mice compared to non-targeted enzyme.

FIG. 15. Graphs showing (A) higher blood levels and (B) lower lung and (C) brain levels of a fusion protein injected as such in mice compared to the same fusion protein loaded in a nanoparticle formulation.

FIG. 16. Graphs showing brain effects of ICAM-1-targeted fusion enzymes in mice following an every other day dosing schedule over a two week period. (A) Sphingomyelin (SM), the substrate of the ASM enzyme, which aberrantly accumulates in the brain of diseased mice (as in humans) was measured to be reduced upon treatment. (B) Esterified cholesterol (CE), which associates to sphingomyelin and also accumulates in the disease, was also measured to be reduced by fusion enzyme treatment. (C) Ceramide (CER), the product of the ASM catalytic reaction, which accumulates and causes side effects by Olipudase®, was determined.

FIG. 17. Graphs showing side effects of ICAM-1-targeted fusion enzymes in mice. (A.) Hematology parameters, (B.) markers of liver and kidney toxicity, (C.) body weight, and (D.) behavior, none showed signs of toxicity.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all nucleotide and amino acid sequences described herein, and every nucleotide sequence referred to herein includes its complementary DNA sequence, and also includes the RNA equivalents thereof, and vice versa. All sequences described herein, whether nucleotide or amino acid, include sequences having 50.0-99.9% identity, inclusive, and including all numbers and ranges of numbers there between to the first decimal point. The identity may be determined across the entire sequence, or a segment thereof that retains its intended function. Homologous sequences from, for example, other enzymes, protease cleavage sites, secretion signals, and targeting moieties, are included within the scope of this disclosure, provided such homologous sequences also retain their intended function. Further, proteins of the present disclosure include functionally equivalent molecules in which amino acid residues are substituted for residues within the sequence resulting in a silent or conservative change. For example, one or more amino acid residues within the sequence can be substituted by another amino acid of a similar polarity that acts as a functional equivalent, resulting in a silent or conservative alteration. Substitutes for an amino acid within the sequence may be selected from other members of the class to which the amino acid belongs. For example, the nonpolar (hydrophobic) amino acids include, but are not limited to, alanine, leucine, isoleucine, valine, proline, phenylalanine, tryptophan, methionine, and combinations thereof. The polar neutral amino acids include, but are not limited to, glycine, serine, threonine, cysteine, tyrosine, asparagine and glutamine, and combinations thereof. The positively charged (basic) amino acids include, but are not limited to, arginine, lysine and histidine. The negatively charged (acidic) amino acids include aspartic acid, glutamic acid, and combinations thereof. Also included within the scope of the disclosure are proteins or fragments or derivatives thereof which exhibit the same or similar biological activity and derivatives which are differentially modified during or after translation, for example, by glycosylation, proteolytic cleavage, and the like.

Any result obtained using a method described herein can be compared to any suitable reference, such as a known value, or a control sample or control value, suitable examples of which will be apparent to those skilled in the art, given the benefit of this disclosure. In embodiments, any result obtained herein can be compared to a value obtained from analysis of components of the fusion proteins described herein, but wherein the components are configured differently, or are present in different copy numbers, or in a different stoichiometry, or are not present in the same, intact polypeptide. In embodiments, the disclosure provides for an improved result, relative to a result obtained using a targeting moiety and an enzyme that are present in the same composition, but are not present in the same polypeptide or were produced in the same polypeptide prior to enzyme release or liberation. In embodiments, the improved result comprises any one or combination of: improved and/or increased enzymatic activity, such as enzyme activity measured at a pH below physiological pH, such as in a lysosome, an improved pharmacokinetic property, improved bioavailability property, improved stability, improved shelf life, improved production yield, improved safety, improved duration of activity, improved biodistribution, improved incorporation into a lysosome, and/or an improved effect on any sign or symptom of a lysosomal storage disease. In embodiments, a result obtained using a composition described herein is improved, relative to a result obtained using a composition that comprises a particulate carrier. In an embodiment, a fusion protein of this disclosure displays increased targeting and/or catalytic activity than a control enzyme that is not a component of a fusion protein. In embodiments, a fusion protein of this disclosure displays a measurable improvement relative to a control enzyme that is not a component of a fusion enzyme. In embodiments, a 1-10 fold improvement is achieved. In non-limiting embodiments, a fusion protein of this disclosure displays ≥700-1000% (7-10-fold) better targeting and/or ≥300% (3-fold) better catalytic activity than a control enzyme such as acid sphingomyelinase (ASM), with ≥50% enhancement after protease cleavage of the enzyme.

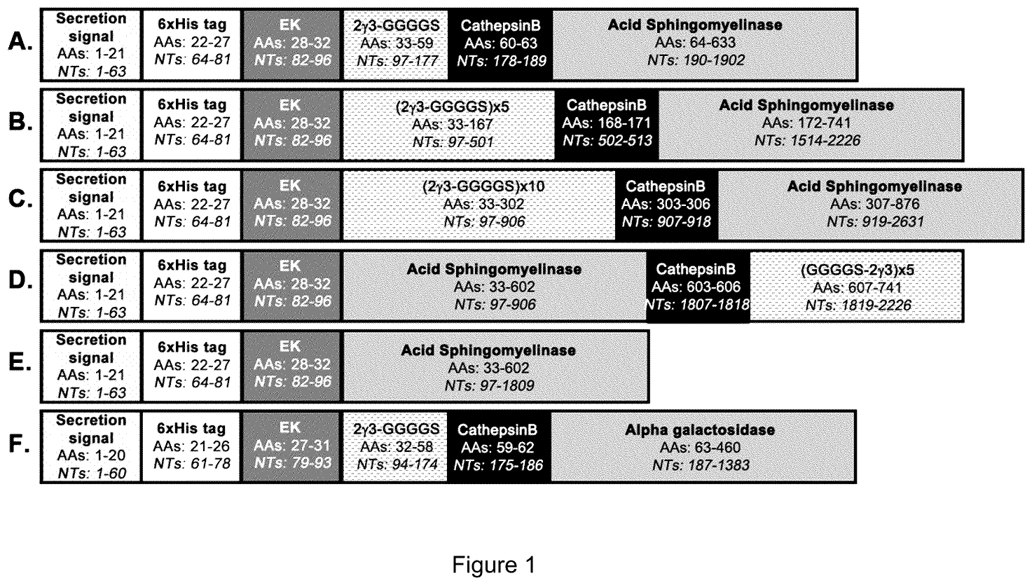

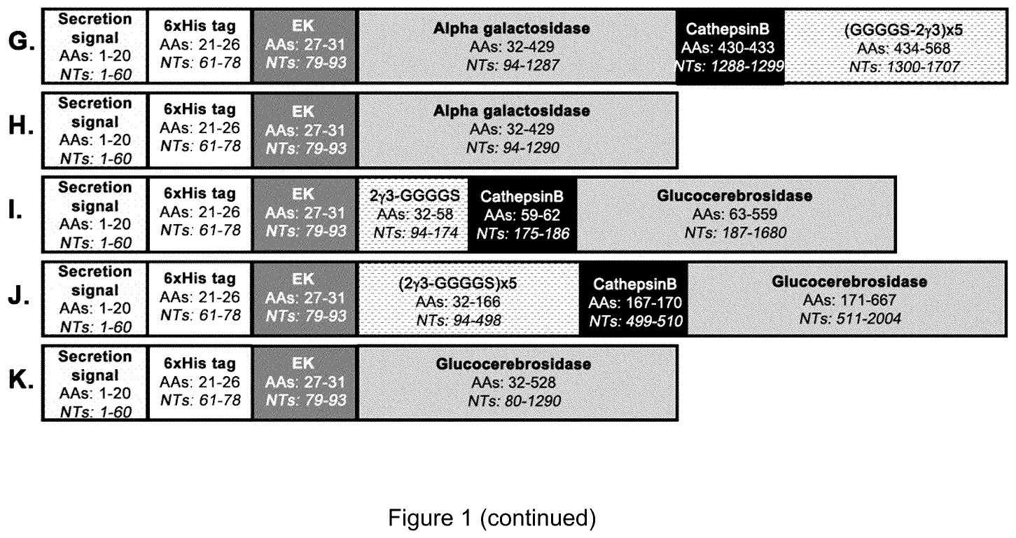

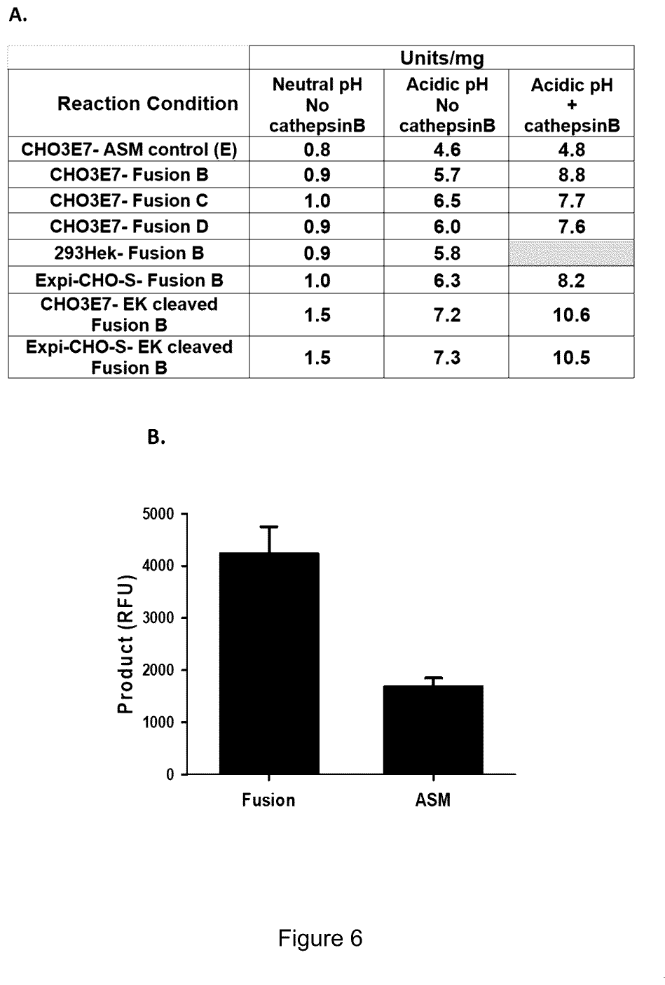

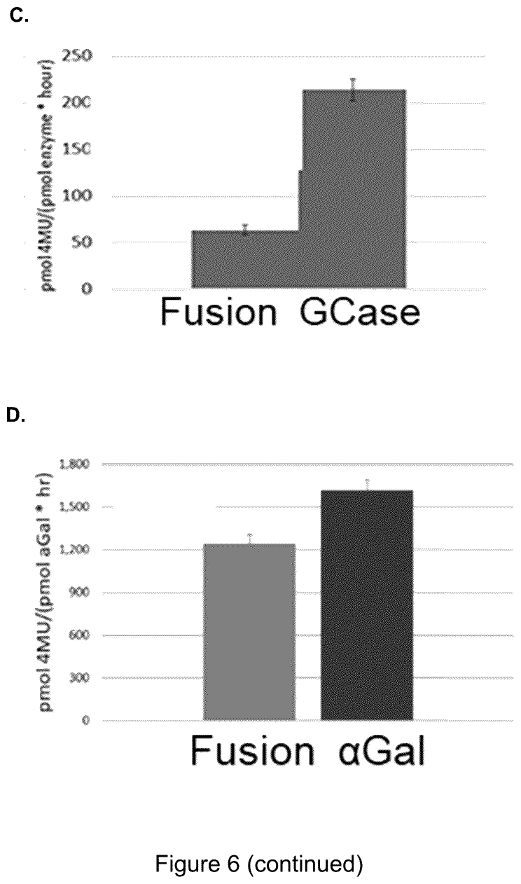

In connection with the foregoing, the present disclosure unexpectedly reveals, as in part demonstrated by Example 6 and FIG. 6 and the data in the Table referred to therein, that after cathepsin B cleavage of the fusion protein, the released enzyme, which no longer contains any other domains but the enzyme, exhibits more activity than an enzyme that was not previously part of a fusion protein. For example, both constructs sequence B and E (said constructs illustrated in FIG. 1) only differ in the targeting peptide and cathepsin B sequence. After cathepsin B cleavage, both products of sequences B and E are no different, yet after cathepsin B cleavage the product of B remains more active than the product of E. Without intending to be bound by any particular theory, it is considered that the fusion protein may have fold differently and, once liberated by protease activity, the enzyme part is more active. Hence, it is considered that the folding of the fusion protein is not the same than the non-fused enzyme. Thus, the disclosure provides for production of a fusion protein that contains a segment that is more active when freed from the fusion protein, relative to the same segment that is used in the absence of the fusion protein. In embodiments, a described fusion protein may therefore be considered to be a prodrug that is suitable for ERT, among other uses.

In one aspect, the disclosure comprises recombinant polypeptides, i.e., fusion proteins, for use in treating one or more LSDs, or additional diseases which may benefit from these enzyme activities, wherein the fusion proteins generally comprises:
i) one or more intercellular adhesion molecule-1 (ICAM-1) targeting segments;
ii) an enzyme segment that is catalytically active at the pH of a lysosome;
iii) optionally a first protease cleavage sequence segment between i) and ii), and optionally, one or more of:
iv) a secretion signal;
v) a protein purification tag; and
vi) a second protease cleavage signal, such as for use in protein purification, for removal of iv) and v) from the final product.

In embodiments, a fusion protein of this disclosure comprises or consists of any combination of i)-vi), provided at least i) and ii), and preferably at least i), ii) and iii) are present.

Representative and non-limiting configurations of segments of fusion proteins that are included in this disclosure are provided in Example 1 and FIG. 1. Representative amino acid sequences of each of these segments, and DNA sequences encoding them, are also provided herein, but are not intended to be limiting. Representative amino acid sequences for constructs A-K in FIG. 1 are provided below as amino acid sequences 13-23, respectively. Numbering in FIG. 1 corresponds with amino acid numbers in the annotated segments of the construct maps.

Where polypeptides of this disclosure are described, expression vectors encoding the polypeptides are also included. The expression vectors can be used in production of the polypeptides, and/or as therapeutic agents, such as DNA vaccines. Representative and non-limiting DNA sequences encoding proteins are provided below.

In embodiments, the ICAM-1 targeting segment comprises or consists of the sequence NNQKIVNIKEKVAQIEA (SEQ ID NO: 1), referred to herein from time to time as 2γ3. In embodiments, the 2γ3 sequence is repeated in the fusion protein. In embodiments, the 2γ3 sequence is repeated 2 to 10 times in the fusion protein. In embodiments, one 2γ3 sequence is proximal to another sequence, such as a Gly and Ser containing sequence. e.g., a linker sequence. In embodiments, a suitable Gly Ser sequence contains GGGGS (SEQ ID NO:24). In embodiments, distinct 2γ3 segments are separated by a segment comprising the sequence GGGGSGGGGS (SEQ ID NO:25). A variety of other linkers are known in the art and can be used with embodiments of this disclosure.

As an alternative to 2γ3, other ICAM-1 targeting peptide sequences can be used. Some examples include but are not necessarily limited to:

(SEQ ID NO: 26)
NNQKIVNLKEKVAQLEA;
(SEQ ID NO: 27)
NNQKLVNIKEKVAQIEA;
(SEQ ID NO: 28)
YPASYQR;
(SEQ ID NO: 29)
YQATPLP;
(SEQ ID NO: 30)
GSLLSAA;
(SEQ ID NO: 31)
FSPHSRT;
(SEQ ID NO: 32)
YPFLPTA
and
(SEQ ID NO: 33)
GCKLCAQ.

In embodiments, a fusion protein described herein comprises a targeting segment and/or a lysosomal enzyme segment described in U.S. Pat. No. 8,778,307, from which the description of targeting moieties and lysosomal enzymes are incorporated herein by reference.

The enzyme segment of the fusion proteins described herein can comprise any enzyme or catalytic fragment thereof that is also described herein. In non-limiting embodiments, the enzyme or catalytic fragment thereof can function in a lysosome. In one embodiment, the enzyme is Acid sphingomyelinase (ASM). In another embodiment, the enzyme is Alpha galactosidase. In another embodiment the enzyme is Glucocerebrosidase (GCase). The amino acid sequences of each of these enzymes are known in the art. As noted above, representative and non-limiting sequences are provided in the examples below, and representative configurations on the enzyme segment in relation to the other components of the fusion proteins are shown in Example 1 and FIG. 1. Any fusion protein of this disclosure may comprise or consist of the sequence of functional fusion proteins depicted in FIG. 1.

In embodiments, the fusion proteins provided by this disclosure comprise a first and second protease cleavage sequence. In general, the first and second protease cleavage sites are distinct from one another, and do not appear elsewhere in the fusion proteins.

In embodiments, the first cleavage sequence comprises a sequence that is cleaved by any protease originally located in a lysosome, which may be located within endosomes or lysosomes, or secreted extracellularly by cells. In embodiments, the protease cleavage signal is cleaved by any endosomal cysteine proteases, such cysteine proteases known in the art as Cathepsins. In embodiments, the protease recognition sequence is recognized by cathepsin L or cathepsin B. In an embodiment, a protease cleavage site used in fusion proteins of this disclosure comprises the sequence GFLG (SEQ ID NO:34). In this regard, representative and non-limiting examples of first protease cleavage site configurations in relation to fusion proteins of this disclosure are shown in Example 1 and FIG. 1, and by way of the sequences provided with this disclosure. In FIG. 1, the represented protease cleavage site is designated as Cathepsin B. The first protease recognition cleavage sequence is configured such that it can liberate the enzyme segment from the remainder of the fusion protein upon cleavage in a suitable physiological solution, such as within a lysosome. In embodiments the protease is thus sequestered to a lysosome, and therefore the enzyme is only liberated subsequent to being taken up by the lysosome. In embodiments, the disclosure thus provides a prodrug that is only activated in the lysosome, or another environment with a sufficiently low pH level such that the protease is active. In embodiments, the prodrug itself has enzymatic activity.

In embodiments, a fusion protein of this disclosure comprises a second protease cleavage signal which is intended to be used in protein isolation and/or purification. In embodiments, the second protease cleavage site is distinct from the first protease cleavage site. In specific but non-limiting embodiments, second protease cleavage sites that can be used in embodiments of this disclosure include the EK cleavage sequence DDDDK (SEQ ID NO: 35), Tobacco etch virus (ENLYFQ (SEQ ID NO:36)), Factor Xa site IEGR (SEQ ID NO: 37), matrix metalloproteinase 9 (MMP-9) PXXXX, where X in position 2 and 3 is any residue, position 3 is a hydrophobic residue, and the X in position 5 is S or T (SEQ ID NO: 38), papain XXXXZRUXXX (SEQ ID NO:39) (where U is any residue but V), and Thrombin LVPRGS (SEQ ID NO:40).

In embodiments, a fusion protein of this disclosure comprises a secretion signal that is used for protein production and/or purification. Any suitable secretion signal can be used and many are known in the art. In one non-limiting embodiments, the secretion signal comprises METDTLLLWVLLLWVPGSTG (SEQ ID NO:41) or MGWSCIILFLVATATGVHSD (SEQ ID NO: 42).

In embodiments, fusion proteins provided in this disclosure may include protein purification tags. Any suitable protein purification tag can be used. In a non-limiting embodiment, a poly-histidine tag is used. A His-tag as used herein is a linear sequence of n histidine residues where n is typically 6-8.

In embodiments, the disclosure comprises administering therapeutically effective amounts of a described fusion protein to an individual in need thereof. In embodiments, the fusion protein to be used in a therapeutic method will have been produced and processed such that the secretion signal and the protein purification tag are removed from a portion of the fusion protein comprising the ICAM-1 targeting and enzyme segments by cleavage at the second protease cleavage sequence. In other embodiments, the purification tag is not removed.

Therapeutically effective amount means that amount of a recombinant polypeptide of this disclosure that will elicit the biological or medical response of a subject that is being sought. A “therapeutically effective amount” in certain implementations means an amount sufficient to prevent or reduce signs and/or symptoms of any disorder wherein an enzyme described herein could have a prophylactic and/or therapeutic benefit.

In embodiments, a therapeutically effective amount rescues an LSD disorder caused by a deficiency of one or more lysosomal enzymes. Effective amounts of polypeptides of this disclosure will depend in part on the particular LSD, the size and weight of the individual, etc. For any recombinant polypeptide disclosed herein, an effective dose can be estimated initially either in cell culture assays or in animal models, usually mice, rabbits, dogs, pigs, or non-human primates. The animal model is also used to achieve a desirable concentration range and route of administration. Such information can then be used to determine useful doses and routes for administration in humans. In embodiments, a fusion protein of this disclosure is administered such that it reaches the lungs and/or brain of an individual. Prophylactic dosing and uses are also included in this disclosure. Prophylactic or therapeutic doses can encompass a broad range of concentration including, but limited to, 0.01 mg/Kg to 20 mg/Kg.

In embodiments, the disclosure provides compositions comprising the described polypeptides, such as pharmaceutical formulations. In embodiments, the non-limiting compositions are free of particulate carriers. In embodiments, compositions are free of any one or combination of polystyrene nanocarriers, poly-lactic co-glycolic acid (PLGA) nanocarriers, polyethylene glycol (PEG), poly-lactic acid (PLA) nanocarriers, and biopolymeric dendrimers. In embodiments, a protein or polynucleotide encoding the protein as provided herein is not covalently or ionically coupled to a particle.

In embodiments, a pharmaceutical composition comprises a pharmaceutically acceptable carrier. Suitable carriers include, for example, diluents, adjuvants, excipients, or other vehicles with which the present complexes may be administered to an individual. Non-limiting examples of materials which can serve as pharmaceutical carriers include: sugars, such as lactose, glucose and sucrose; starches, such as corn starch and potato starch; cellulose, including sodium carboxymethyl cellulose, ethyl cellulose and cellulose acetate; powdered tragacanth; malt; gelatin; talc; excipients, such as cocoa butter; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The composition, if desired, can also contain minor amounts of wetting or emulsifying agents, or pH buffering agents. Some examples of compositions suitable for mixing a composition of this disclosure can be found in: Remington: The Science and Practice of Pharmacy (2005) 21st Edition, Philadelphia, Pa. Lippincott Williams & Wilkins.

Methods of using the therapeutic compositions include administration by any acceptable approaches including but not limited orally and parenterally. For example, the recombinant polypeptides can be administered intramuscularly, subdermally, subcutaneously, topically, intracranially, intratracheally, by instillation, intravenously, and intra-arterial. In embodiments, the fusion protein is loaded on or in cells for release in the body, e.g., cells such as erythrocytes can be loaded with proteins, injected in circulation, and release the protein over time which then target the intended organs. In embodiments, a fusion protein of the disclosure is administered in combination with any suitable nanoparticles. In embodiments, a composition comprising a fusion protein may be administered by a device, such as a medical pump, implant, patch, or chip.

In embodiments, a polypeptide or polynucleotide encoding such polypeptide is administered to an individual in need thereof. In embodiments, the individual in need thereof has been diagnosed with or is suspected of having any disorder that is correlated with a lysosomal storage disease, non-limiting examples of which include Pompe Disease, GM1 gangliosidosis, Tay-Sachs disease, GM2 gangliosidosis, Sandhoff disease, Fabry disease, Gaucher disease, metachromatic leukodystrophy, Krabbe disease, Niemann-Pick disease type A, Niemann-Pick disease type B, Niemann-Pick disease type C, Niemann-Pick disease type D, Farber disease, Wolman disease, Hurler Syndrome, Scheie Syndrome, Hurler-Scheie Syndrome, Hunter Syndrome, Sanfilippo A Syndrome, Sanfilippo B Syndrome, Sanfilippo C Syndrome, Sanfilippo D Syndrome, Morquio A disease, Morquio B disease, Maroteaux-Lamy disease, Sly Syndrome, α-mannosidosis, β-mannosidosis, fucosidosis, aspartylglucosaminuria, sialidosis, mucolipidosis II, mucolipidosis III, mucolipidosis IV, Goldberg Syndrome, Schindler disease, cystinosis, Salla disease, infantile sialic acid storage disease, Batten disease, infantile neuronal ceroid lipofuscinosis, or prosaposin.

In alternative embodiments, the individual has any type of cancer, or Parkinson's disease. In embodiments, the cancer is a blood cancer or a solid tumor, either primary or metastatic, that affects any part of the body.

The disclosure includes expression vectors encoding the fusion proteins, and methods of making the fusion protein using the expression vectors. In general, a method of making the fusion proteins comprises allowing expression of an expression vector encoding the fusion proteins in a cell culture, and separating the protein from the cell culture using any suitable approach. The proteins can be separated and purified to any desired degree of purity. In certain embodiments, the disclosure includes cell cultures that contain the expression vectors. In certain embodiments, the cell cultures are eukaryotic cell cultures.

In embodiments, the disclosure includes administering to an individual an expression vector encoding a fusion protein described herein, or otherwise configured to result in expression of the fusion protein once the expression vector is introduced into a cell. In embodiments, any of a variety of retroviral vectors, such as lentiviral vectors, or adenoviruses, adeno-associated viruses, herpes, and vaccinia viruses can be used. In embodiments, RNA encoding the fusion protein can be directly injected into the cells or otherwise introduced to the individual. Polynucleotide vectors can include modified nucleotides or phosphate backbone moieties, many suitable examples of which are known in the art. CRISPR/Cas technology can also be used to introduce in the genome the coding sequence of the described fusion proteins.

In one representative approach, which is further illustrated by the Examples presented below, 5 tandem repeats of 2γ3 were cloned for enhanced ICAM-1 affinity, each repeat separated from the next by a short peptide linker to enable their independent folding (targeting domain=135 amino acids). At the carboxyl-terminus of the targeting domain, a 4 amino acid cathepsin B sequence was placed to enable lysosomal cleavage and release of human ASM, which was cloned at the carboxyl-terminus of the release domain. This catalytic domain is 570 amino acids-long and encompasses human ASM mRNA sequence starting at His62, which lacks the enzyme's natural secretion sequence. The targeting+cleavage+catalytic cassette (“functional domains”) was preceded at the amino-terminus by “production domains”, consisting of a 21 amino acid signal peptide for secretion of the fusion protein from transfected cells into the culture medium, a 6 amino acid His-tag for affinity purification, and a 5 amino acid enterokinase cleavage site to separate the production domains from the functional domains which constitute the intended fusion protein. The full sequence was cloned into a plasmid vector to enable transient and stable expression in mammalian cells. In addition, computer predictions were pursued to examine the resulting conformation of the targeted fusion proteins. Results suggested that the designed fusion protein would match the folding of respective human wild-type enzymes which endogenously reside in lysosomes of our bodies.

To produce data described in the Examples below, a plasmid vector containing the expression cassette for an ASM fusion protein was transfected in CHO-3E7, Expi-CHO-S and 293-6E cells. In all cases, collection of the cell medium 5-7 days post-transfection, followed by affinity purification using the His-tag domain and electrophoresis under reducing conditions, showed a protein band of ≈80 KDa (expected size), quantified as ≈90% purity. Similar cells were transfected with plasmids containing the expression cassettes for GCase and αGal fusion proteins, respectively, which also resulted in the production of proteins of expected size and purity ranging from 39 to 90%. Subsequent optimization of the purification protocol raised purity to ≥90% in all cases. Electrophoresis under non-reducing conditions revealed monomeric, dimeric, and tetrameric protein at ≈25%, 25%, 50% for 293 cells and 30%, 15%, 55% for CHO cells in the case of ASM fusion protein. This was because natural ASM assembles into monomeric and oligomeric forms in the body, all of which are functional. Three independent production rounds in CHO cells showed high reproducibility. The production yield was high, e.g. ≈5-8 mg/L of culture for CHO-3E7 and ≈35-40 mg/mL for Expi-CHO-S. Western blot (WB) validated that the fusion protein contained His-tag and enzyme modules, for which antibodies are available. It also validated that the His-tag was cleaved off the fusion protein by enterokinase, which was then removed by size exclusion chromatography for a final purity of ≈85%. In the case of an ASM fusion protein, WB also showed that cathepsin B cleavage released a protein band of ≈70 KDa recognized by anti-ASM, as expected. Similar results were obtained for GCase fusion protein and αGal fusion protein.

When hundreds of copies of γ3 peptides are coupled on the surface of polymer nanoparticles, particles target human and mouse ICAM-1 (Garnacho et al., J Pharm Exp Ther, 340:638 & Garnacho et al., J Drug Targeting. 25:786). However, these peptides were never tested outside the context of these high affinity nanoparticles. We observed that, in CHO cells that expressed 2γ3-ASM fusion protein, ASM was stained using anti-ASM+FITC−secondary antibody, abundant label associated with the cells. This was in contrast to non-transfected CHO cells, despite the fact that CHO cells express natural ASM, which we verified by Western blotting (WB). Fluorescence detection could have been enabled by enhanced expression of fusion protein over natural ASM, yet we had demonstrated that fusion protein is secreted to the cell medium. In fact, WB validated the almost absence of fusion protein in cell lysates vs. the cell medium. Therefore, the fluorescence signal may be due to binding of secreted fusion protein on cells, indicating ICAM-1 binding, as WB verified the presence of ICAM-1 expression in these CHO cells. In addition, incubation of purified fusion protein with HUVEC cells (known to express ICAM-1) for 3 h at 37° C., followed by cell fixation, permeabilization and staining using anti-ASM+FITC-secondary antibody, showed bright green-FITC dots indicative of endo-lysosomal uptake of the fusion protein by cells. This was not observed when HUVECs were incubated with control non-targeted ASM (Olipudase), demonstrating the targeting ability of the fusion protein.

After demonstrating the presence of an ASM enzyme sequence in the fusion protein, its catalytic function was assessed. For this purpose, a commercial kit was used, which is based on catalytic hydrolysis of sphingomyelin and final production of fluorescent Resorufin, which can be quantified by spectrofluorometry. As further demonstrated in the Examples below, in the absence of cathepsin B or low cathepsin B concentration, some ASM activity was found over negative control levels, indicating that the fusion protein is active. More importantly, ASM activity doubled upon raising cathepsin B concentration, indicating that a fully active enzyme results upon release by cathepsin B. Furthermore, ASM activity increased by several fold at pH 4.5, which is reflective of lysosomal conditions, vs. neutral pH 7.4. These results suggest that maximal ASM activity will be obtained upon lysosomal delivery of the fusion protein, which would prevent any undesirable ASM activity in circulation, observed for Olipudase. Similar results were found for GCase and αGal fusion proteins, indicating that all fusion proteins have prevalent activity under lysosomal conditions. In addition, the catalytic activity of the fusion protein under lysosomal conditions was 2-3-fold enhanced compared to the control ASM. The activity of GCase and αGal fusion proteins was also enhanced compared to their respective controls.

Next, both the targeting and trafficking of fusion proteins were compared against similar control non-targeted enzymes. For example, in a pharmacological model of ASM deficiency, cell association of control ASM was much lower (7-10 fold by immunofluorescence detection and 3-4 fold by radioisotopic tracing) compared to ASM fusion protein. Similarly, association of GCase fusion protein to induced pluripotent stem cell (iPS)-derived neurons from a Gaucher patient was about 3 fold enhanced compared to respective control non-targeted enzyme. In the latter case, GCase fusion was visualized to traffic to lysosomes in neurons, while control Cerezyme® used at similar activity units was not visible.

Skin fibroblasts from patients diagnosed for ASM deficiency (NPD) and skin fibroblasts from healthy individuals were tested as a part of this disclosure. No personal data was associated with them. First, both healthy and diseased cells were incubated overnight with a commercial fluorescent sphingomyelin (BODIPY-FL-C12-sphingomyelin), a substrate analogue for ASM, which fluoresces green. Microscopy examination of said cells showed that diseased cells accumulated increased levels of sphingomyelin compared to healthy cells. Incubation of diseased cells for 5 h with control, non-targeted ASM (from which Olipudase was derived) vs. similar concentration of fusion protein resulted in differential degradation of the stored sphingomyelin. Control ASM only degraded 4% of the sphingomyelin stored in diseased cell vs. 27% degradation for the fusion protein, which represents a 6-7-fold improvement in the intracellular activity after only 5 h incubation. Similar results were found for GCase fusion protein and αGal fusion proteins when compared to respective control enzymes in skin fibroblasts from patient with Gaucher disease and Fabry disease, respectively.

Apart from enhanced enzymatic activity and substrate reduction observed by fusion proteins, additional effects were studied. For instance, fluorescence microscopy showed that acidic compartments such as lysosomes were aberrantly engorged in iPS-derived neurons from Gaucher patients compared to healthy wildtype counterparts. Incubation with GCase fusion protein normalized the size of said compartments while control Cerezyme exerted only a partial reduction. In addition, GCase fusion protein did not cause cytotoxicity after 48 incubation with iPS-derived neurons compared to a positive control, H₂O₂, which is known to cause cell death.

Next, the capacity of fusion proteins to be transported across the BBB was tested in a multicellular model consisting of human brain endothelial cells, human astrocytes and iPS-derived neurons from a Gaucher patient. After validating the barrier function of this model, GCase fusion protein was demonstrated to cross this BBB model and accumulate in the subjacent neurons, while control non-targeted enzyme was trapped in the BBB and did not significantly accumulate in neurons after 24 h incubation. Additionally, pre-incubation of this model with anti-ICAM antibody blocked the interaction of GCase fusion with cells, while pre-incubation with anti-mannose-6-phosphate receptor antibody did not. This demonstrated an ICAM-1, not mannose-6-phosphate receptor, mediated process.

The ASM knock-out mouse mimics both type A (neurological) and type B (peripheral) NPD. We radiolabeled samples and injected i.v. 0.13 mg/Kg of ¹²⁵I-ASM-fusion protein or ¹²⁵I-ASM in mice. Measurement of the radiotracer in blood and tissues showed that both proteins disappeared fast from the circulation: by 1 h, 20% of the injected dose (% ID) was in blood for ASM and only 8.5% ID for the ASM-fusion protein. Since Olipudase has shown systemic toxicity, a reduction in circulation time for ASM-fusion protein may improve this. ASM-fusion protein was detected in the brain, lung, liver, spleen, heart, and kidneys, all of which need treatment. The localization ratio, which is the tissue-to-blood accumulation (% ID per gram in an organ over % ID per gram in the blood), was increased for the ASM-fusion protein over control ASM even after only 1 h after one single dose: e.g., 35% increase in the brain (main target in type A NPD) and 80% increase in the lung, 3.3-fold in the liver, and 3-fold in the spleen (main targets in type B NPD). Hence, the fusion protein had enhanced in vivo delivery. In addition, this fusion protein was loaded on nanoparticles, which showed enhanced removal from the circulation and enhanced targeting to peripheral organs (e.g. the lungs) and the central nervous system (e.g. the brain) compared to fusion protein not loaded in nanoparticles. Next, mice were injected i.v. with 0.6 mg/kg of ASM-fusion protein without nanoparticles, every two days for a total of 6 injections, vs. mice injected with control buffer. At the end of the experiment, blood and organs were measured for sphingomyelin and cholesterol, disease hallmarks. Multiple sphingomyelin and cholesterol species were reduced, which is needed for therapy. Ceramide product (associated to Olipudase side effects) was not significantly increased. An example is shown below for the brain, the organ where Olipudase has no effect. As seen, in the Examples below, 10 sphingomyelin species and 16 cholesterol species were lowered upon treatment with ASM-fusion protein. Instead, only a ceramide species was slightly increased upon treatment which suggest lack of any major ceramide burst which may lead to relevant side effects.

Mice were monitored each day during the study. The mice showed no statistical changes in the body weight for ASM-fusion protein vs. control buffer, or for parameters such as grooming and general activity. Hematological (RBCs, all types of leukocytes, platelets) and biochemical (glucose) tests showed no statistically significant changes between mice injected with fusion protein vs. control buffer, and this was also true for renal toxicity markers (BUN, creatinine) and hepatic toxicity markers (alkaline phosphatase). This, together with no overt increase in ceramide, the ASM product which is burst-produced and leads to toxicity of current ASM-Olipudase ERT, shows relative safety of the presently provided fusion strategy.

It will be apparent to those skilled in the art that the foregoing description illustrates: 1) fusion proteins of this disclosure have been generated and encompass various configurations of a 2γ3 ICAM-1 targeting module and ASM, GCase, or αGal catalytic modules, separated by a cathepsin B cleavable peptide which leads to the release of functional enzyme within the lysosomes; (2) these fusion protein possess enhanced targeting, trans-BBB transport, cellular uptake, and lysosomal trafficking in pharmacological cell models and patient cells; and (3) fusion proteins provide enhanced catalytic activity under lysosomal conditions in vitro and in cell cultures, in comparison to control non-targeted enzymes and commercial enzymes; (4) they provide enhanced substrate reduction and lysosomal size reduction; and (5) these fusion proteins surpassed both the targeting and functional performance, with respect to control enzyme, in mouse models (particularly the brain), with no appreciable side effects.

The foregoing results are reiterated and expanded upon by the following Examples, which are presented in order to more fully illustrate the preferred embodiments of the invention and should in no way be construed as limiting the scope of the invention. Unless indicated otherwise, for representative demonstrations described in these

Examples, controls correspond to the cDNA or amino acid sequence of non-targeted enzymes, while all other cases represent the cDNA or amino acid sequence of fusion proteins consisting of an ICAM-1 targeting domain and an enzyme domain, separated by a cleavage domain to release the enzyme domain from the targeting domain in the lysosome. In all cases, control and others, the cDNA or amino acid sequences may contain at the amino terminus of the proteins a signal peptide domain for secretion, followed by a tag domain for purification, followed by a domain for cleavage of said signal and tag domains.

### EXAMPLES

Example 1, illustrated by FIG. 1, Expression cassette of ICAM-1-targeted fusion enzymes. Schematics of the domain design for: (A) Human acid sphingomyelinasc (ASM) with one copy of the 2γ3 ICAM-1-targeting peptide at the amino terminus; (B) human ASM with five tandem-repeats of the 2γ3 ICAM-1-targeting peptide at the amino terminus; (C) human ASM with ten tandem-repeats of the 2γ3 ICAM-1-targeting peptide at the amino terminus; (D) human ASM with five tandem-repeats of the 2γ3 ICAM-1-targeting peptide at the carboxyl terminus; (E) human ASM control; (F) human alpha galactosidase (αGal) with one copy of the 2γ3 ICAM-1-targeting peptide at the amino terminus; (G) human αGal with five tandem-repeats of the 2γ3 ICAM-1-targeting peptide at the carboxyl terminus; (H) human αGal control; (I) human glucocerebrosidase (GCase) with one copy of the 2γ3 ICAM-1-targeting peptide at the amino terminus; (J) human GCase with five tandem-repeats of the 2γ3 ICAM-1-targeting peptide at the amino terminus; and (K) human GCase control. Controls correspond to the non-targeted enzymes, while all other cases represent fusion proteins consisting of a targeting domain and an enzyme domain, separated by a cathepsin B cleavage domain to release enzyme domain from the targeting domain in the lysosome. In all cases, control and others, the expression cassettes contain at the amino terminus of the fusion proteins a signal peptide domain for secretion, followed by a His-tag domain for purification, followed by an enterokinase domain (EK) for cleavage of said signal and tag domains. Also, in all cases, human enzymes are truncated at their amino termini to eliminate endogenous signal peptides. From these amino acid (AAs) designs, the corresponding nucleotide (NTs) designs were made using codon optimization for the intended expression in mammalian cells.

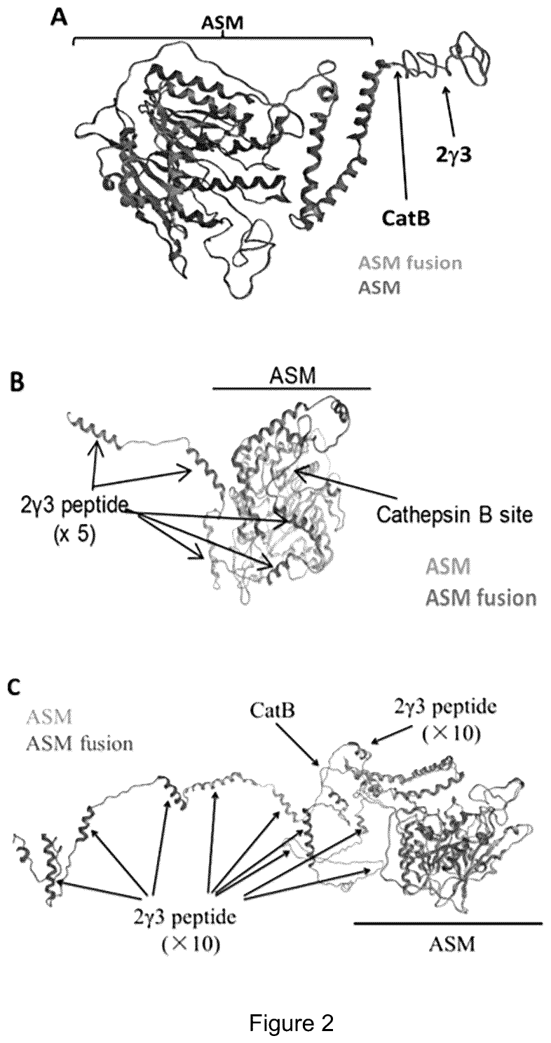

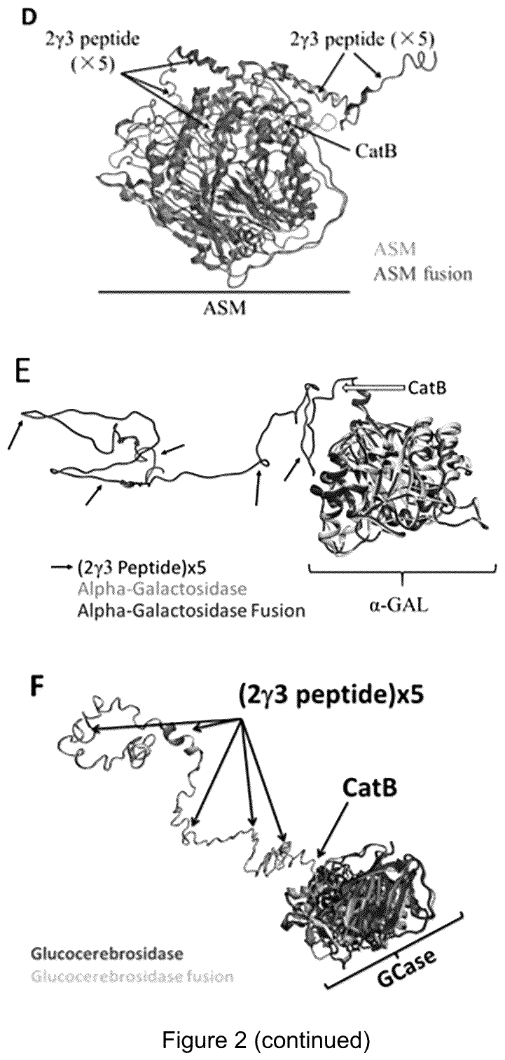

Example 2, illustrated by FIG. 2. Predicted structure of ICAM-1-targeted fusion enzymes. A) Fusion protein containing one copy of the 2γ3 ICAM-1-targeting peptide at the amino terminus, followed by a cathepsin B (CatB) cleavage site for lysosomal release of truncated human acid sphingomyelinase (ASM). (B) Fusion protein containing five tandem-repeats of the 2γ3 ICAM-1-targeting peptide at the amino terminus, followed by CatB cleavage site for lysosomal release of truncated human ASM. (C) Fusion protein containing ten tandem-repeats of the 2γ3 ICAM-1-targeting peptide at the amino terminus, followed by CatB cleavage site for lysosomal release of truncated human ASM. (D) Fusion protein containing truncated human ASM at the amino terminus, followed by CatB cleavage site for lysosomal release, followed by five tandem-repeats of the 2γ3 ICAM-1-targeting peptide at the carboxy terminus. (E) Fusion protein containing truncated human alpha galactosidase (αGal) at the amino terminus, followed by CatB cleavage site for lysosomal release, followed by five tandem-repeats of the 2γ3 ICAM-1-targeting peptide at the carboxy terminus. (F) Fusion protein containing five tandem-repeats of the 2γ3 ICAM-1-targeting peptide at the amino terminus, followed by CatB cleavage site for lysosomal release of truncated human glucocerebrosidase (GCase).

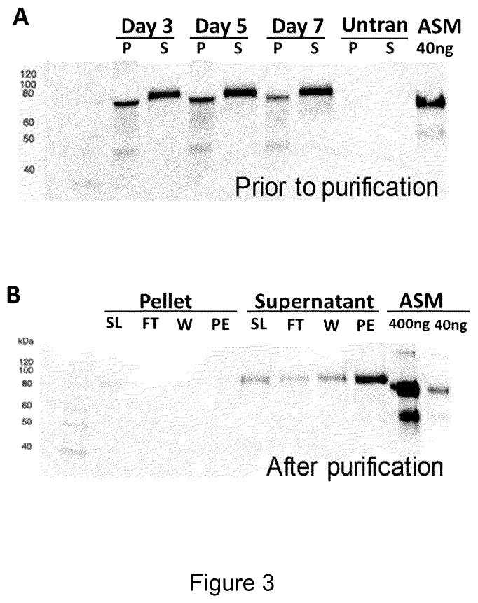

Example 3, illustrated by FIG. 3. Production and purification of ICAM-1-targeted fusion enzymes. Protein electrophoresis followed by western blotting of fusion proteins secreted by CHO cells, shown (A) prior and (B) after fusion protein affinity purification via His-tag domain. Fusion protein is that shown in panel (B) in Example 2 above. (A) Higher molecular weight band in the cell medium supernatant(S) corresponds to the fusion protein, versus the low molecular weight band in the cell pellet (P), which corresponds to the cell endogenous enzyme. ASM=control purified recombinant enzyme has lower molecular weight than fusion protein and similar to endogenous ASM in cells. Different production days are shown. (B) No fusion protein is purified from the cell pellet fractions (Sample (SL), Flow-through after passing through an anti-his tag affinity column (FT), wash fraction (W), and pooled elution from the affinity column (PE)), since endogenous ASM does not have a His-tag. Fusion protein is present in the corresponding fractions from the cell medium supernatant, verifying its secretion. The pooled eluted fraction of the cell medium supernatant is enriched for the fusion protein. Untran=untransfected control cells.

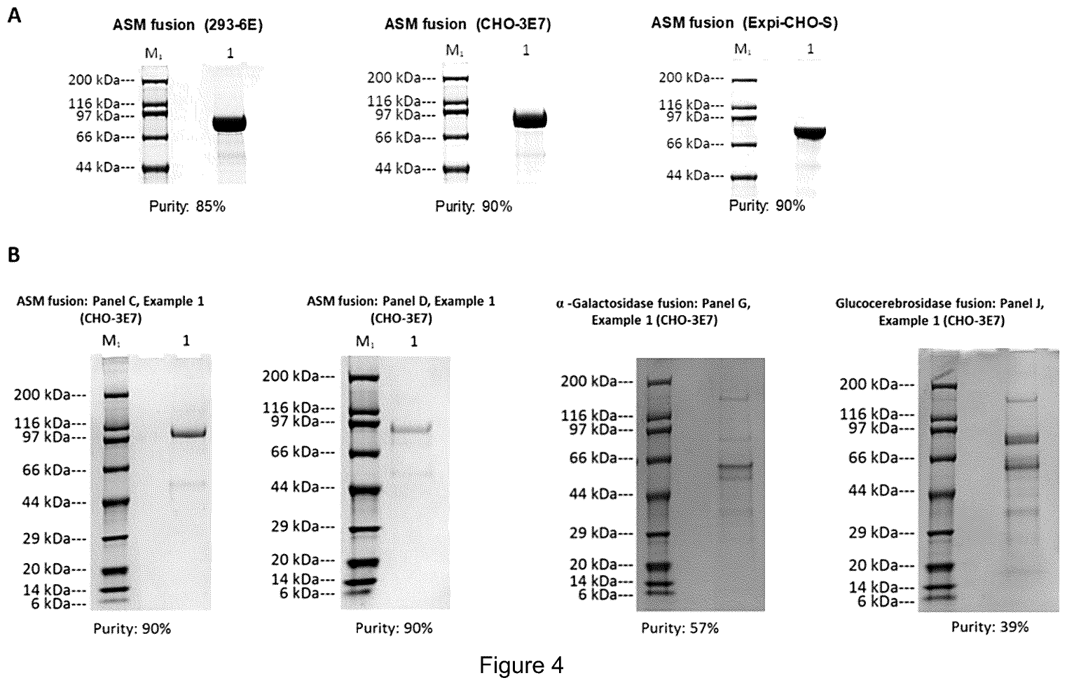

Example 4, illustrated by FIG. 4. Reproducibility and purity of ICAM-1-targeted fusion enzymes. (A) Electrophoresis followed by Coomassie blue staining demonstrate that the fusion protein in panel (B) in Example 2 above was independently produced in and purified from three mammalian cell lines (293-6E, CHO-3E7, and Expi-CHO-S), showing great reproducibility and purity. (B) Other fusion proteins are shown, including fusion proteins in panels C, D, G and J (Example 1 above) all produced in the CHO-3E7 cell line.

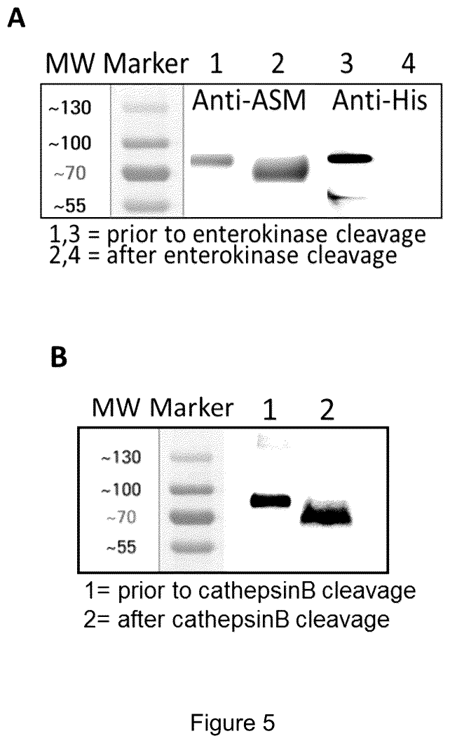

Example 5, illustrated by FIG. 5. Western blotting of ICAM-1-targeted fusion enzymes. (A) After purification of the fusion protein shown in panel (B) in Example 2 above, Western blotting was conducted using antibody to detect the enzyme domain (ASM) and the His-tag domain used for purification, both prior and after enterokinase cleavage to remove the His-tag from the fusion protein. (B) Similarly, the purified fusion protein shown in panel (B) in Example 2 above was cleaved with cathepsin B to remove the targeting group from the enzyme and western blotting was conducted to determine the enzyme domain (ASM). The lower molecular weight band corresponds to that of the ASM portion of the fusion protein.

Example 6, illustrated by FIG. 6. Enzymatic activity of ICAM-1-targeted fusion enzymes. (A) The table shows the comparative in vitro enzymatic activity of fusion proteins and respective non-fusion enzyme control as well as some enterokinase (EK) cleaved fusion proteins, prior and after release with cathepsin B, at lysosomal versus neutral pH, and for fusions produced in different cell lines where B, C, D and E are as in Example 1. (B) Enzymatic activity, under lysosomal conditions, of fusion protein B in Example 1 was additionally compared to full recombinant ASM produced by He et al. (He, Miranda et al. 1999), which served as basis for Genzyme Olipudase®. The fusion enzymes are more active in lysosomal conditions (acidic) compared to circulation conditions (neutral) and are even more active after removal of the His tag, as expected. (C) Enzymatic activity, under lysosomal conditions, of GCase fusion protein J in Example 1 compared to control non-targeted GCase protein K in Example 1. (D) Enzymatic activity, under lysosomal conditions, of α-Gal fusion protein G in Example 1 compared to control non-targeted α-Gal protein H in Example 1.

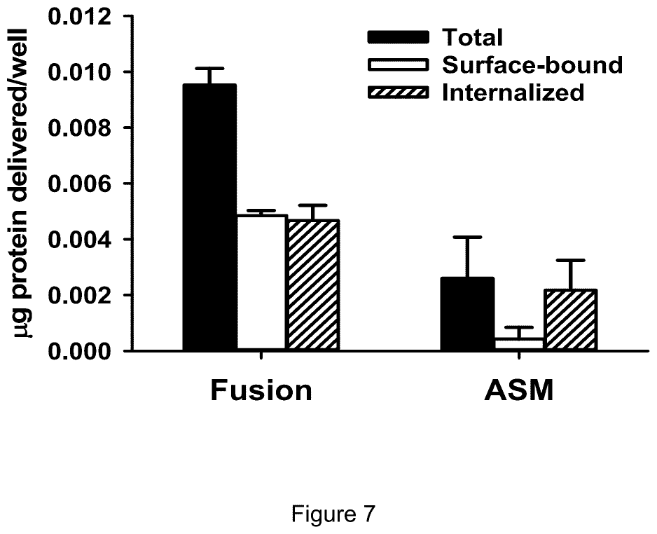

Example 7, illustrated by FIG. 7. Cell binding and internalization of ICAM-1-targeted fusion enzymes. A pharmacological model of Niemann-Pick disease types A and B was used, which consists of treating cells with imipramine, a small molecule known to degrade endogenous ASM. Cells were additionally treated with TNFα to mimic an inflammatory status, as it pertains to Niemann-Pick disease. Cells were then incubated for 3 hours at 37° C. with either fusion protein B in Example 1 (after enterokinase cleavage), or full recombinant ASM produced by He et al. (He, Miranda et al. 1999), which served as basis for Genzyme Olipudase®. In both cases, proteins were labeled with ¹²⁵Iodine to allow tracing of their association with cells. Surface-bound fraction was eluted with a glycine solution and the remaining, non-eluted fraction corresponds to internalized protein. The sum of both fractions represents the total cell association.

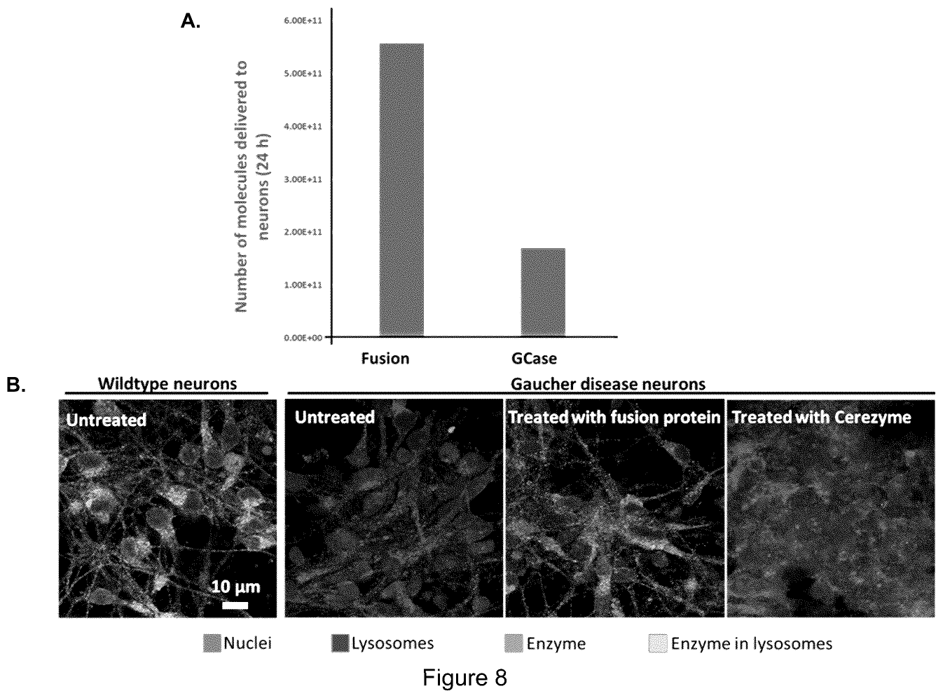

Example 8, Illustrated by FIG. 8. Uptake and lysosomal trafficking of ICAM-1-targeted fusion enzymes by neurons. (A) Induced pluripotent stem cells (iPS)-derived neurons bearing mutations from a Gaucher disease patient and treated with TNFα to mimic an inflammatory status, were incubated for 24 h at 37° C. with either targeted fusion GCase protein J in Example 1 or with control non-targeted GCase protein K in Example 1 (both after enterokinase cleavage). In both cases, these proteins had been pre-labeled with ¹²⁵Iodine to trace them. The number of molecules associated to cells was quantified using a gamma counter to measure the radioactive label. (B) iPS-derived neurons bearing the wildtype GCase sequence, or, bearing Gaucher disease mutations and treated with TNFα to mimic an inflammatory status, were fixed, permeabilized and stained using fluorescently-labeled antibodies to detect lysosomes (anti-Lamp1) in red color and GCase enzyme (anti-GCase) in green color. Lysosomal trafficking of these proteins appears in green+red=yellow-orange color. Cell nuclei was stained in blue using DAPI. The same procedure was used for mutant neurons after 24 h treatment with either targeted fusion GCase protein or control non-targeted Cerezyme, a commercial recombinant GCase. Scale bar=10 μm.

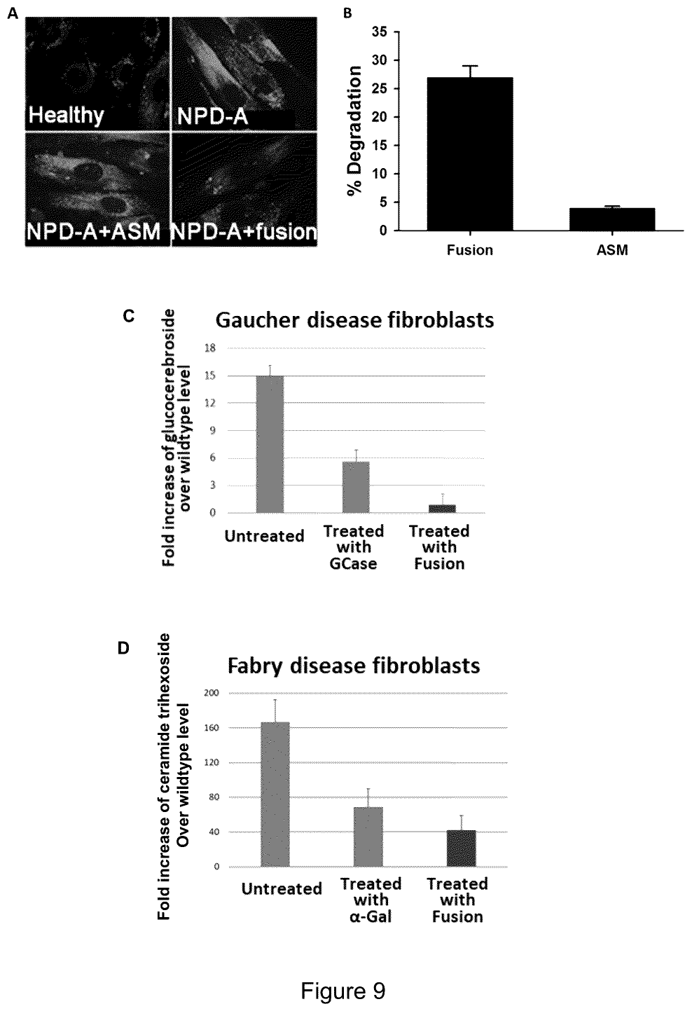

Example 9, illustrated by FIG. 9. Reduction of lysosomal storage in patient cells by ICAM-1-targeted fusion enzymes. (A) Sphingomyelin labeling with BODIPY-FL-C12-sphingomyelin in cultured fibroblasts from healthy versus Niemann-Pick type A patient cells, prior to or after incubation with the same dose (16.7 g/mL) of fusion ASM or non-fusion control. Sphingomyelin aberrantly accumulated in patient cells, since this is the substrate of ASM, which is deficient in these patients. (B) Quantification of the level of BODIPY-FL-C12-sphingomyelin degraded by fusion protein or non-fusion control delivered to patient cells, showing increased therapeutic degradation of the substrate by the fusion protein. (C) Fibroblasts from a Gaucher disease patient were incubated with fluorescent N-hexanoyl-NBD-glucosylceramide to visualize the accumulation of this lipid due to disease, and then left untreated or treated for 5 h with either targeted GCase fusion protein J from Example 1 or control non-targeted GCase protein K from Example 1 (both after enterokinase cleavage). The level of fluorescent N-hexanoyl-NBD-glucosylceramide in wildtype fibroblasts was also visualized and normalized to 1, so that the lipid level in untreated or treated diseased cells was compared to wild-type levels (fold increase). (D) A similar experiment to (C) is shown, yet this time tracing the accumulation of fluorescent N-Dodecanoyl-NBD-ceramide trihexoside in wildtype fibroblasts and fibroblasts from a Fabry disease patient that were either not treated or treated with α-Gal fusion protein G from Example 1 or control non-targeted α-Gal protein H from Example 1 (both after enterokinase cleavage).

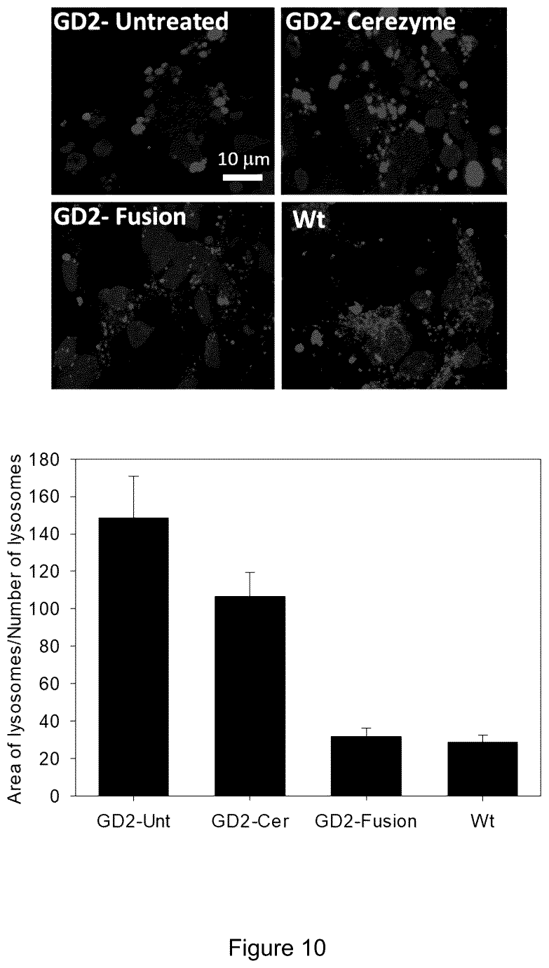

Example 10, illustrated by FIG. 10. Attenuation of the enlargement of lysosomes in diseased neurons by ICAM-1-targeted fusion enzymes. (A) Induced pluripotent stem cells (iPS)-derived neurons bearing wildtype GCase sequence (Wt) or bearing mutations from a Gaucher disease patient (GD2) were treated with TNFα to mimic an inflammatory status. Then, cells were left untreated (GD2-Unt) or were incubated for 24 h at 37° C. with ether targeted fusion GCase protein J in Example 1 (after enterokinase cleavage) or with commercially available Cerezyme. Lysotracker was used to label lysosomes with red fluorescence and cells were fixed. Microscopy was finally used to image lysosomes and quantify their average size (area they occupy per cell/number of lysosomal vesicles per cell).

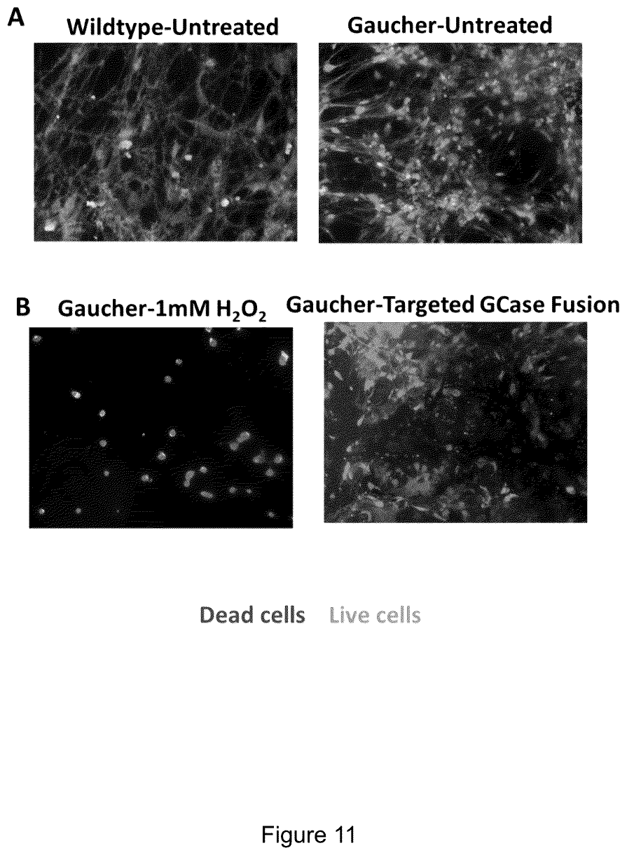

Example 11, illustrated by FIG. 11. Lack of cytotoxicity of ICAM-1-targeted fusion proteins. (A) Induced pluripotent stem cells (iPS)-derived neurons bearing wildtype GCase sequence or bearing Gaucher patient mutations were treated with TNFα overnight to mimic an inflammatory status. The number of live cells or dead cells were visualized using a live/dead viability assay where calcein stains the cytoplasm of live cells green while ethidium homodimer stains dead cell nuclei red, respectively. (B) Similarly, neurons bearing Gaucher patient mutations were incubated with 1 mM H₂O₂ for 1 h to induce cell death as a control or for 48 h with targeted GCase fusion protein J in example 1 (after enterokinase cleavage), then the same live/dead assay was used.

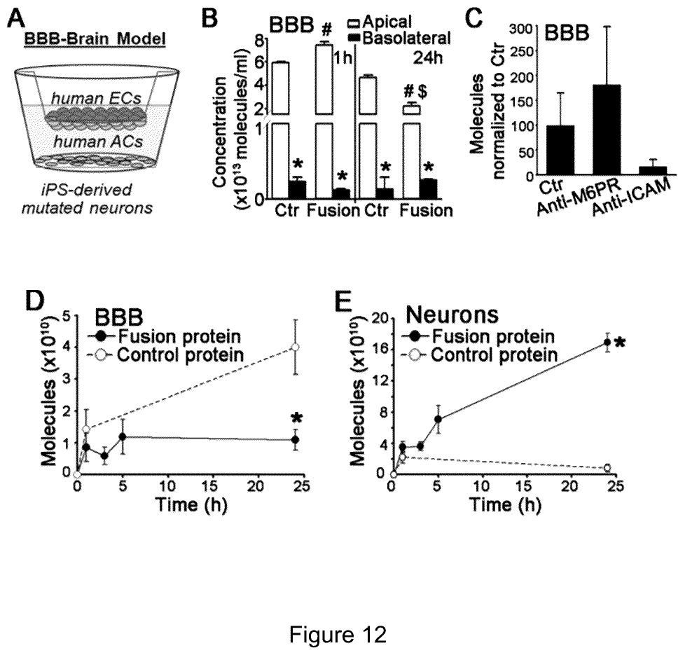

Example 12, illustrated by FIG. 12. Transcytosis of ICAM-1-targeted fusion enzymes across models of the blood-brain barrier and uptake by subjacent neurons. (A) Transwell model of the blood-brain barrier formed by human brain endothelial cells growing on the apical side of a porous filter, astrocytes growing on the basolateral side of the same filter, and these two cellular monolayers separating an apical chamber (mimicking the blood vessel side) from a basolateral chamber (mimicking the brain tissue side). These cells were treated with conduritol-β-epoxide to mimic a Gaucher disease phenotype. Induced pluripotent stem cells (iPS)-derived neurons bearing mutations from a Gaucher disease patient were grown on the bottom of the basolateral chamber. Cells were additionally treated with TNFα to mimic an inflammatory status typical of this disease. (B) ICAM-1 targeted fusion GCase protein J (example 1; after enterokinase cleavage) or control (Ctr) non-targeted GCase protein K (example 1; after enterokinase cleavage) were pre-labeled with ¹²⁵Iodine for tracing purposes and added to the apical chamber above the BBB for 1 h or 24 h. After this time, the amount of proteins in the apical or in the basolateral chambers was quantified. The graph shows the concentration of protein molecules left in either chamber, demonstrating the lack of free diffusion or leakage across this BBB model, which can thus be considered a good barrier model. (C) The amount of targeted fusion GCase that interacted with the BBB was quantified after 3 h and compared to the amount of targeted fusion GCase interacting the BBB when cells had been pre-incubated with anti-mannose-6-phosphate receptor or anti-ICAM receptor to block the respective receptor. (D) Presence of fusion GCase protein or control GCase in the BBB or (E) basolateral iPS-neurons over time. Data are average±SEM, * p<0.05 (Student's t-test).

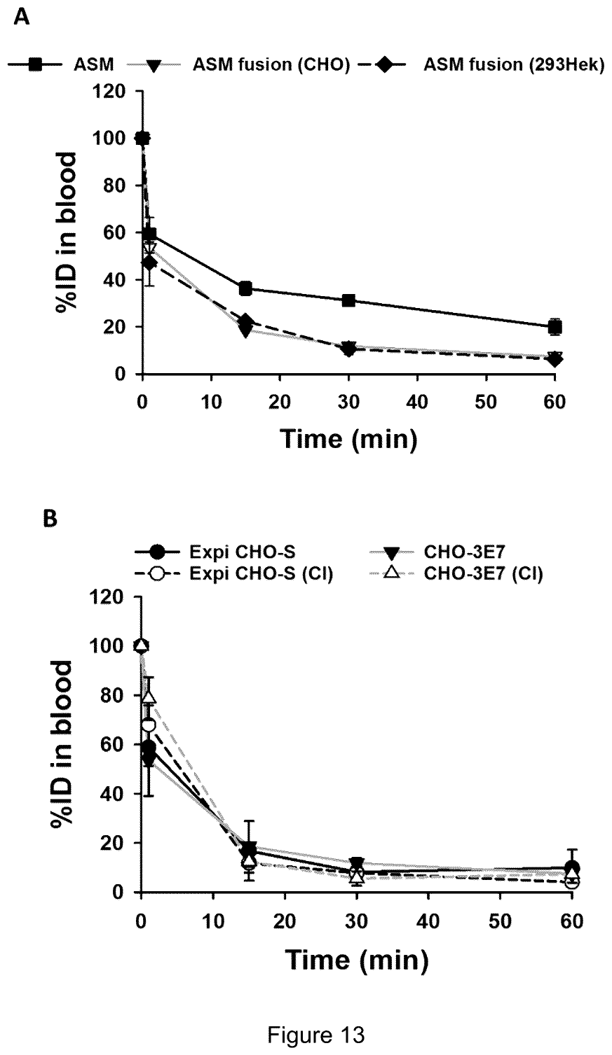

Example 13, illustrated by FIG. 13. Circulation of ICAM-1-targeted fusion enzymes in mice. Blood levels of proteins labeled with ¹²⁵Iodine, expressed as a percentage of the injected dose (0.13 mg/Kg), determined at the indicated times after their intravenous injection in ASM knockout mice, the model for Niemann-Pick disease type A and B. (A) Fusion protein B in Example 1, produced from two different cell sources (CHO-3E7 versus Hek 293 cells) is compared to full recombinant ASM produced by He et al. (He, Miranda et al. 1999), which served as basis for Genzyme's Olipudase®. Faster disappearance of fusion proteins is expected due to targeting to tissues, and should be beneficial in lowering systemic side effects and resistance due to immunorecognition. (B) Circulation of the same fusion in two different CHO cell lines, before and after cleavage (Cl) of the His-tag domain by enterokinasc.

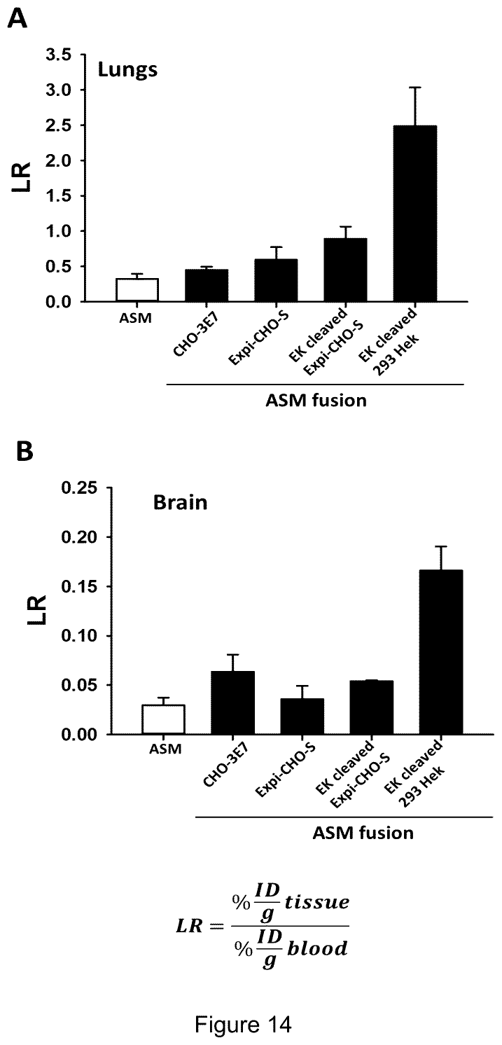

Example 14, illustrated by FIG. 14. Lung and brain distribution of ICAM-1-targeted fusion enzymes in mice. (A) Lung and (B) brain levels of proteins labeled with ¹²⁵Iodine, expressed as the localization ratio (LR), 60 minutes after intravenous injection of 0.13 mg/Kg in mice (lung and brain are main targets for Niemann-Pick disease type B and A, respectively). Fusion protein B in Example 1, produced from three cell sources (CHO-3E7, Expi-CHO-S versus Hek 293 cells), prior or after cleavage with enterokinase (EK) to remove His-tag, is compared to full recombinant ASM produced by He et al. (He, Miranda et al. 1999), which served as basis for Genzyme's Olipudase®. Enhanced targeting is shown for all fusion protein.

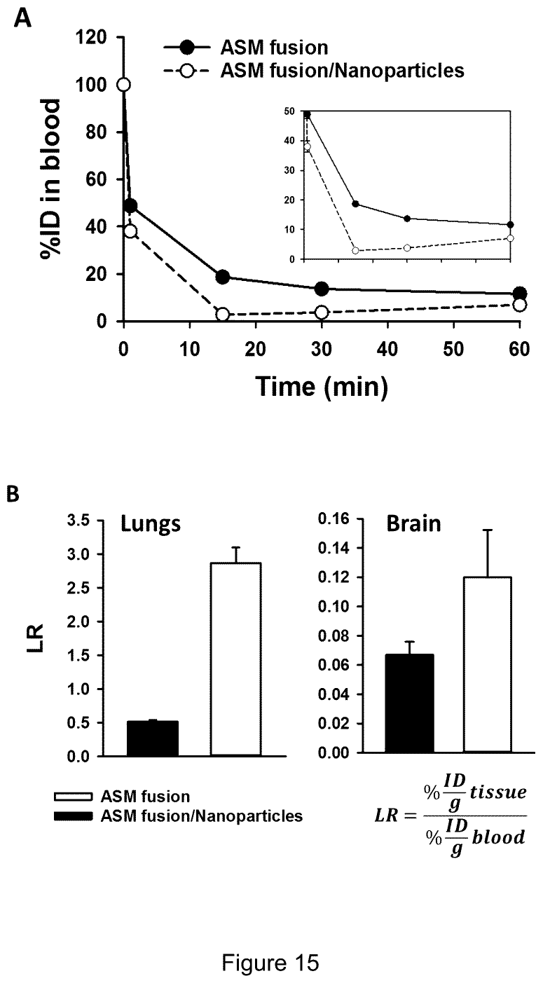

Example 15, illustrated by FIG. 15. Lung and brain distribution of ICAM-1-targeted fusion enzymes administered in mice as nanoparticle formulations. (A) Blood levels of a “naked” fusion protein compared to a fusion protein loaded in a nanoparticle formulation, determined at the indicated times after their intravenous injection and expressed as a percentage of the injected dose (% ID) in blood. The nanoparticle formulation had faster disappearance (the inset shows a close up of the large graph for additional detail), which is expected due to the increase targeting to tissues (see B) and should be beneficial in lowering any potential systemic side effects of the fusion protein. and resistance due to immunorecognition. (B) Lung and brain levels of “naked” versus nanoparticle-loaded fusion protein, expressed as the localization ratio (LR) found 60 min after injection (lung and brain are main targets for Niemann-Pick disease type B and A, respectively). The nanoparticle formulation showed 5-6 fold enhanced lung accumulation and 2-fold enhanced brain accumulation.

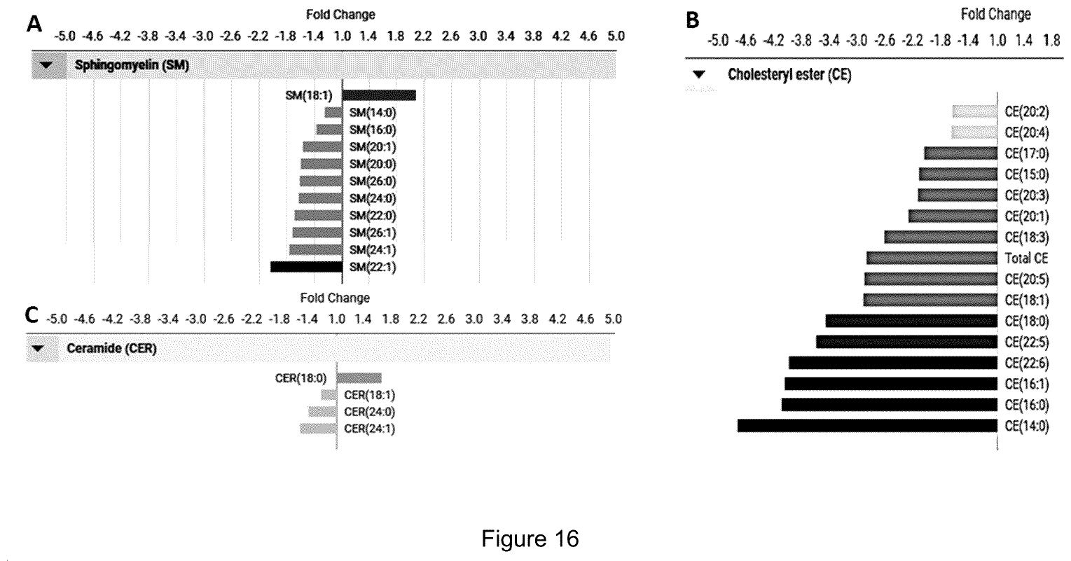

Example 16, illustrated by FIG. 16. Brain effects of ICAM-1-targeted fusion enzymes in mice. ASM knockout mice were injected with 0.6 mg/Kg of enterokinasc cleaved fusion protein B in Example 1 every two days, for a total of 6 injections, and were compared to mice injected with vehicle buffer but no fusion protein (sham control). At the end of the experiment, (A) sphingomyelin (SM), the substrate of the ASM enzyme, which aberrantly accumulates in the brain of diseased mice (as in humans) was measured. (B) Cholesterol (CE), which associates to sphingomyelin and also accumulates in the disease, was also measured. (C) Ceramide (CER), the product of the ASM catalytic reaction, which accumulates and causes side effects by Olipudase®, was determined. In all cases, increases of SM, CE, or CER are marked as a positive fold change (bar on the right of the middle line), while decreases are marked as a negative fold change (bars on the left of the middle line). Middle lines are non-treated diseased controls. Both SM and CE were significantly lowered, without dangerous.

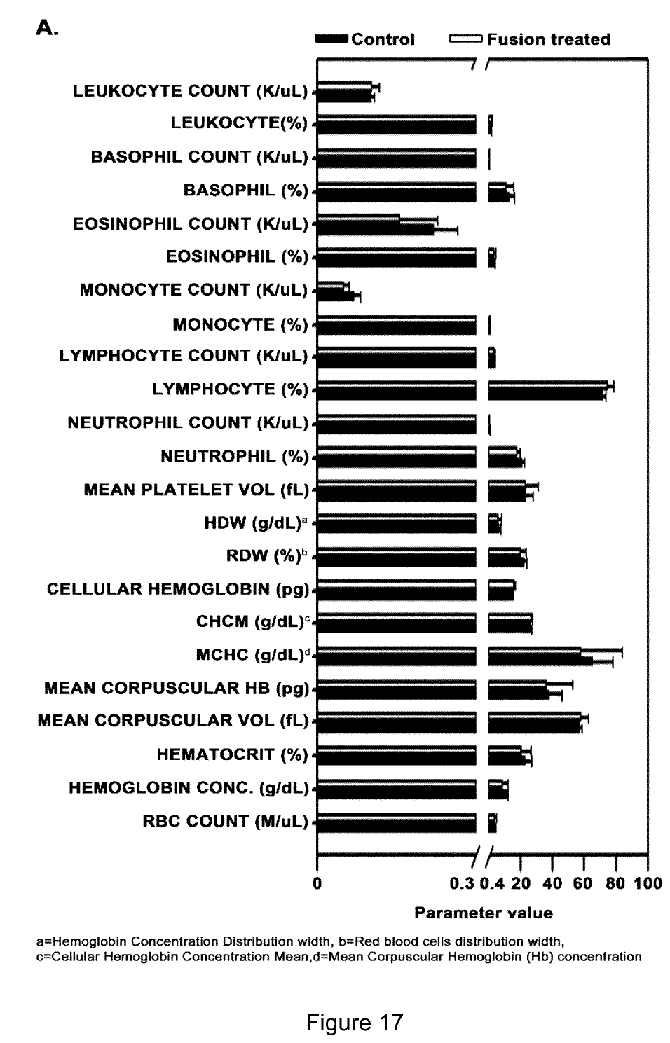

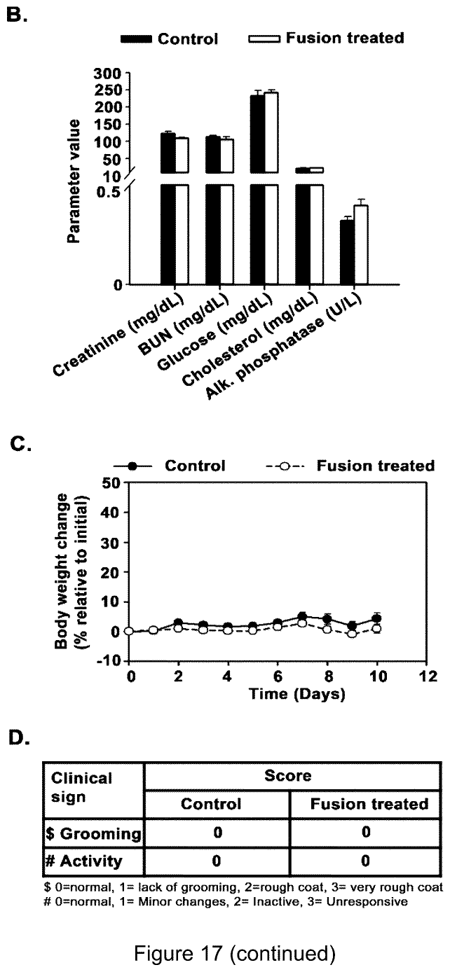

Example 17, illustrated by FIG. 17. Side effects of ICAM-1-targeted fusion enzymes in mice. The same animals shown in example 16 were examined for side effects, including: (A) hematology parameters, (B) markers of liver and kidney toxicity, (C) body weight, and (D) behavior. In terms of all the parameters tested, treatment with the fusion protein did not induce toxicity compared to the sham treated group (control). It will be recognized from the foregoing description and figures that the strategy described herein provides improved results in peripheral organs, which are the main target for type B NPD and many other LSDs. This is expected to help lower the dose required for therapeutic activity in these organs with concomitant decrease in cost and side effects, which benefits both drug manufacturers and patients, and can be extended from NPD to all current lysosomal ERTs used for peripheral organ treatment. In addition, the present fusion protein strategy exhibits enhanced targeting and measurable functional effects in the brain, a non-peripheral organ of the central nervous system where no current lysosomal ERT can reach. Thus, this strategy represents a breakthough in the treatment of type A NPD and is applicable to ≈40 additional LSDs with neurological syndromes. Lastly, unlike previous nanoparticulate formulations, which involved polymeric materials that have never been approved for chronic use in pediatric patients, the fusion protein platform described herein can be produced by classical biotechnological means, as done for current ERTs approved by FDA. Reproducibility, high yield and purity, and versatility of production in different cells supports manufacturing and reduces regulatory hurdles for implementing embodiments of the disclosure.

The following sequences are representative and non-limiting examples of embodiments of the disclosure, and relate to the constructs depicted in FIG. 1, and to the results described herein.

### Sequence 1. Amino Acid Sequence of the ICAM-1 Targeting Segment, 2γ3.

(SEQ ID NO: 1)
NNQKIVNIKEKVAQIEA

Sequence 2. cDNA sequence of the expression cassette for human acid sphingomyelinase (ASM) with one copy of the 2γ3 ICAM-1-targeting peptide at the amino terminus

(SEQ ID NO: 2)
ATGGAGACCGACACACTGCTCCTGTGGGTCCTGCTCCTCTGGGTGCCAGG
AAGTACAGGAGATCACCATCACCATCACCACGACGACGACGACAAGAATA
ACCAAAAGATTGTGAATATCAAAGAGAAAGTGGCTCAGATTGAGGCTGGA
GGCGGAGGAAGCGGCGGCGGAGGAAGCGGATTTCTGGGACACCCTCTTTC
TCCCCAAGGCCATCCTGCCAGGTTACATCGCATAGTGCCCCGGCTCCGAG
ATGTCTTTGGGTGGGGGAACCTCACCTGCCCAATCTGCAAAGGTCTATTC
ACCGCCATCAACCTCGGGCTGAAGAAGGAACCCAATGTGGCTCGCGTGGG
CTCCGTGGCCATCAAGCTGTGCAATCTGCTGAAGATAGCACCACCTGCCG
TGTGCCAATCCATTGTCCACCTCTTTGAGGATGACATGGTGGAGGTGTGG
AGACGCTCAGTGCTGAGCCCATCTGAGGCCTGTGGCCTGCTCCTGGGCTC
CACCTGTGGGCACTGGGACATTTTCTCATCTTGGAACATCTCTTTGCCTA
CTGTGCCGAAGCCGCCCCCCAAACCCCCTAGCCCCCCAGCCCCAGGTGCC
CCTGTCAGCCGCATCCTCTTCCTCACTGACCTGCACTGGGATCATGACTA
CCTGGAGGGCACGGACCCTGACTGTGCAGACCCACTGTGCTGCCGCCGGG
GTTCTGGCCTGCCGCCCGCATCCCGGCCAGGTGCCGGATACTGGGGCGAA
TACAGCAAGTGTGACCTGCCCCTGAGGACCCTGGAGAGCCTGTTGAGTGG
GCTGGGCCCAGCCGGCCCTTTTGATATGGTGTACTGGACAGGAGACATCC
CCGCACATGATGTCTGGCACCAGACTCGTCAGGACCAACTGCGGGCCCTG
ACCACCGTCACAGCACTTGTGAGGAAGTTCCTGGGGCCAGTGCCAGTGTA
CCCTGCTGTGGGTAACCATGAAAGCACACCTGTCAATAGCTTCCCTCCCC
CCTTCATTGAGGGCAACCACTCCTCCCGCTGGCTCTATGAAGCGATGGCC
AAGGCTTGGGAGCCCTGGCTGCCTGCCGAAGCCCTGCGCACCCTCAGAAT
TGGGGGGTTCTATGCTCTTTCCCCATACCCCGGTCTCCGCCTCATCTCTC
TCAATATGAATTTTTGTTCCCGTGAGAACTTCTGGCTCTTGATCAACTCC
ACGGATCCCGCAGGACAGCTCCAGTGGCTGGTGGGGGAGCTTCAGGCTGC
TGAGGATCGAGGAGACAAAGTGCATATAATTGGCCACATTCCCCCAGGGC
ACTGTCTGAAGAGCTGGAGCTGGAATTATTACCGAATTGTAGCCAGGTAT
GAGAACACCCTGGCTGCTCAGTTCTTTGGCCACACTCATGTGGATGAATT
TGAGGTCTTCTATGATGAAGAGACTCTGAGCCGGCCGCTGGCTGTAGCCT
TCCTGGCACCCAGTGCAACTACCTACATCGGCCTTAATCCTGGTTACCGT
GTGTACCAAATAGATGGAAACTACTCCGGGAGCTCTCACGTGGTCCTGGA
CCATGAGACCTACATCCTGAATCTGACCCAGGCAAACATACCGGGAGCCA
TACCGCACTGGCAGCTTCTCTACAGGGCTCGAGAAACCTATGGGCTGCCC
AACACACTGCCTACCGCCTGGCACAACCTGGTATATCGCATGCGGGGCGA
CATGCAACTTTTCCAGACCTTCTGGTTTCTCTACCATAAGGGCCACCCAC
CCTCGGAGCCCTGTGGCACGCCCTGCCGTCTGGCTACTCTTTGTGCCCAG
CTCTCTGCCCGTGCTGACAGCCCTGCTCTGTGCCGCCACCTGATGCCAGA
TGGGAGCCTCCCAGAGGCCCAGAGCCTGTGGCCAAGGCCACTGTTTTGC
TAG

Sequence 3. cDNA sequence of the expression cassette for human ASM with five tandem-repeats of the 2γ3 ICAM-1-targeting peptide at the amino terminus.

(SEQ ID NO: 3)
ATGGAGACCGACACACTGCTCCTGTGGGTCCTGCTCCTCTGGGTGCCAGG
AAGTACAGGAGATCACCATCACCATCACCACGACGACGACGACAAGAATA
ACCAAAAGATTGTGAATATCAAAGAGAAAGTGGCTCAGATTGAGGCTGGA
GGCGGAGGAAGCGGCGGCGGAGGAAGCAATAATCAGAAAATCGTCAACAT
TAAGGAAAAGGTCGCCCAGATTGAAGCAGGAGGCGGCGGCAGCGGCGGAG
GCGGAAGCAATAATCAGAAGATTGTTAACATCAAAGAAAAGGTGGCCCAA
ATTGAAGCAGGAGGAGGAGGATCTGGAGGCGGAGGCAGCAATAACCAGAA
GATCGTCAACATCAAGGAAAAGGTGGCTCAGATCGAGGCAGGAGGCGGAG
GAAGCGGAGGGGGCGGCTCTAACAACCAGAAAATCGTGAACATCAAAGAG
AAAGTGGCTCAGATCGAAGCCGGCGGAGGAGGATCCGGAGGAGGAGGAAG
CGGATTTCTGGGACACCCTCTTTCTCCCCAAGGCCATCCTGCCAGGTTAC
ATCGCATAGTGCCCCGGCTCCGAGATGTCTTTGGGTGGGGGAACCTCACC
TGCCCAATCTGCAAAGGTCTATTCACCGCCATCAACCTCGGGCTGAAGAA
GGAACCCAATGTGGCTCGCGTGGGCTCCGTGGCCATCAAGCTGTGCAATC
TGCTGAAGATAGCACCACCTGCCGTGTGCCAATCCATTGTCCACCTCTTT
GAGGATGACATGGTGGAGGTGTGGAGACGCTCAGTGCTGAGCCCATCTGA
GGCCTGTGGCCTGCTCCTGGGCTCCACCTGTGGGCACTGGGACATTTTCT
CATCTTGGAACATCTCTTTGCCTACTGTGCCGAAGCCGCCCCCCAAACCC
CCTAGCCCCCCAGCCCCAGGTGCCCCTGTCAGCCGCATCCTCTTCCTCAC
TGACCTGCACTGGGATCATGACTACCTGGAGGGCACGGACCCTGACTGTG
CAGACCCACTGTGCTGCCGCCGGGGTTCTGGCCTGCCGCCCGCATCCCGG
CCAGGTGCCGGATACTGGGGCGAATACAGCAAGTGTGACCTGCCCCTGAG
GACCCTGGAGAGCCTGTTGAGTGGGCTGGGCCCAGCCGGCCCTTTTGATA
TGGTGTACTGGACAGGAGACATCCCCGCACATGATGTCTGGCACCAGACT
CGTCAGGACCAACTGCGGGCCCTGACCACCGTCACAGCACTTGTGAGGAA
GTTCCTGGGGCCAGTGCCAGTGTACCCTGCTGTGGGTAACCATGAAAGCA
CACCTGTCAATAGCTTCCCTCCCCCCTTCATTGAGGGCAACCACTCCTCC
CGCTGGCTCTATGAAGCGATGGCCAAGGCTTGGGAGCCCTGGCTGCCTGC
CGAAGCCCTGCGCACCCTCAGAATTGGGGGGTTCTATGCTCTTTCCCCAT
ACCCCGGTCTCCGCCTCATCTCTCTCAATATGAATTTTTGTTCCCGTGAG
AACTTCTGGCTCTTGATCAACTCCACGGATCCCGCAGGACAGCTCCAGTG
GCTGGTGGGGGAGCTTCAGGCTGCTGAGGATCGAGGAGACAAAGTGCATA
TAATTGGCCACATTCCCCCAGGGCACTGTCTGAAGAGCTGGAGCTGGAAT
TATTACCGAATTGTAGCCAGGTATGAGAACACCCTGGCTGCTCAGTTCTT
TGGCCACACTCATGTGGATGAATTTGAGGTCTTCTATGATGAAGAGACTC
TGAGCCGGCCGCTGGCTGTAGCCTTCCTGGCACCCAGTGCAACTACCTAC
ATCGGCCTTAATCCTGGTTACCGTGTGTACCAAATAGATGGAAACTACTC
CGGGAGCTCTCACGTGGTCCTGGACCATGAGACCTACATCCTGAATCTGA
CCCAGGCAAACATACCGGGAGCCATACCGCACTGGCAGCTTCTCTACAGG
GCTCGAGAAACCTATGGGCTGCCCAACACACTGCCTACCGCCTGGCACAA
CCTGGTATATCGCATGCGGGGCGACATGCAACTTTTCCAGACCTTCTGGT
TTCTCTACCATAAGGGCCACCCACCCTCGGAGCCCTGTGGCACGCCCTGC
CGTCTGGCTACTCTTTGTGCCCAGCTCTCTGCCCGTGCTGACAGCCCTGC
TCTGTGCCGCCACCTGATGCCAGATGGGAGCCTCCCAGAGGCCCAGAGCC
TGTGGCCAAGGCCACTGTTTTGCTAG

Sequence 4. cDNA sequence of the expression cassette for human ASM with ten tandem-repeats of the 2γ3 ICAM-1-targeting peptide at the amino terminus.

(SEQ ID NO: 4)
ATGGAGACCGACACACTGCTCCTGTGGGTCCTGCTCCTCTGGGTGCCAGG
AAGTACAGGAGATCACCATCACCATCACCACGACGACGACGACAAGAATA
ACCAAAAGATTGTGAATATCAAAGAGAAAGTGGCTCAGATTGAGGCTGGA
GGCGGAGGAAGCGGCGGCGGAGGAAGCAATAATCAGAAAATCGTCAACAT
TAAGGAAAAGGTCGCCCAGATTGAAGCAGGAGGCGGCGGCAGCGGCGGAG
GCGGAAGCAATAATCAGAAGATTGTTAACATCAAAGAAAAGGTGGCCCAA
ATTGAAGCAGGAGGAGGAGGATCTGGAGGCGGAGGCAGCAATAACCAGAA
GATCGTCAACATCAAGGAAAAGGTGGCTCAGATCGAGGCAGGAGGCGGAG
GAAGCGGAGGGGGCGGCTCTAACAACCAGAAAATCGTGAACATCAAAGAG
AAAGTGGCTCAGATCGAAGCCGGCGGAGGAGGATCCGGAGGAGGAGGAAG
CAATAACCAAAAGATTGTGAATATCAAAGAGAAAGTGGCTCAGATTGAGG
CTGGAGGCGGAGGAAGCGGCGGCGGAGGAAGCAATAATCAGAAAATCGTC
AACATTAAGGAAAAGGTCGCCCAGATTGAAGCAGGAGGCGGCGGCAGCGG
CGGAGGCGGAAGCAATAATCAGAAGATTGTTAACATCAAAGAAAAGGTGG
CCCAAATTGAAGCAGGAGGAGGAGGATCTGGAGGCGGAGGCAGCAATAAC
CAGAAGATCGTCAACATCAAGGAAAAGGTGGCTCAGATCGAGGCAGGAGG
CGGAGGAAGCGGAGGGGGCGGCTCTAACAACCAGAAAATCGTGAACATCA
AAGAGAAAGTGGCTCAGATCGAAGCCGGCGGAGGAGGATCCGGAGGAGGA
GGAAGCGGATTTCTGGGACACCCTCTTTCTCCCCAAGGCCATCCTGCCAG
GTTACATCGCATAGTGCCCCGGCTCCGAGATGTCTTTGGGTGGGGGAACC
TCACCTGCCCAATCTGCAAAGGTCTATTCACCGCCATCAACCTCGGGCTG
AAGAAGGAACCCAATGTGGCTCGCGTGGGCTCCGTGGCCATCAAGCTGTG
CAATCTGCTGAAGATAGCACCACCTGCCGTGTGCCAATCCATTGTCCACC
TCTTTGAGGATGACATGGTGGAGGTGTGGAGACGCTCAGTGCTGAGCCCA
TCTGAGGCCTGTGGCCTGCTCCTGGGCTCCACCTGTGGGCACTGGGACAT
TTTCTCATCTTGGAACATCTCTTTGCCTACTGTGCCGAAGCCGCCCCCCA
AACCCCCTAGCCCCCCAGCCCCAGGTGCCCCTGTCAGCCGCATCCTCTTC
CTCACTGACCTGCACTGGGATCATGACTACCTGGAGGGCACGGACCCTGA
CTGTGCAGACCCACTGTGCTGCCGCCGGGGTTCTGGCCTGCCGCCCGCAT
CCCGGCCAGGTGCCGGATACTGGGGCGAATACAGCAAGTGTGACCTGCCC
CTGAGGACCCTGGAGAGCCTGTTGAGTGGGCTGGGCCCAGCCGGCCCTTT
TGATATGGTGTACTGGACAGGAGACATCCCCGCACATGATGTCTGGCACC
AGACTCGTCAGGACCAACTGCGGGCCCTGACCACCGTCACAGCACTTGTG
AGGAAGTTCCTGGGGCCAGTGCCAGTGTACCCTGCTGTGGGTAACCATGA
AAGCACACCTGTCAATAGCTTCCCTCCCCCCTTCATTGAGGGCAACCACT
CCTCCCGCTGGCTCTATGAAGCGATGGCCAAGGCTTGGGAGCCCTGGCTG
CCTGCCGAAGCCCTGCGCACCCTCAGAATTGGGGGGTTCTATGCTCTTTC
CCCATACCCCGGTCTCCGCCTCATCTCTCTCAATATGAATTTTTGTTCCC
GTGAGAACTTCTGGCTCTTGATCAACTCCACGGATCCCGCAGGACAGCTC
CAGTGGCTGGTGGGGGAGCTTCAGGCTGCTGAGGATCGAGGAGACAAAGT
GCATATAATTGGCCACATTCCCCCAGGGCACTGTCTGAAGAGCTGGAGCT
GGAATTATTACCGAATTGTAGCCAGGTATGAGAACACCCTGGCTGCTCAG
TTCTTTGGCCACACTCATGTGGATGAATTTGAGGTCTTCTATGATGAAGA
GACTCTGAGCCGGCCGCTGGCTGTAGCCTTCCTGGCACCCAGTGCAACTA
CCTACATCGGCCTTAATCCTGGTTACCGTGTGTACCAAATAGATGGAAAC
TACTCCGGGAGCTCTCACGTGGTCCTGGACCATGAGACCTACATCCTGAA
TCTGACCCAGGCAAACATACCGGGAGCCATACCGCACTGGCAGCTTCTCT
ACAGGGCTCGAGAAACCTATGGGCTGCCCAACACACTGCCTACCGCCTGG
CACAACCTGGTATATCGCATGCGGGGCGACATGCAACTTTTCCAGACCTT
CTGGTTTCTCTACCATAAGGGCCACCCACCCTCGGAGCCCTGTGGCACGC
CCTGCCGTCTGGCTACTCTTTGTGCCCAGCTCTCTGCCCGTGCTGACAGC
CCTGCTCTGTGCCGCCACCTGATGCCAGATGGGAGCCTCCCAGAGGCCCA
GAGCCTGTGGCCAAGGCCACTGTTTTGCTAG

Sequence 5. cDNA sequence of the expression cassette for human ASM with five tandem-repeats of the 2γ3 ICAM-1-targeting peptide at the carboxyl terminus.

(SEQ ID NO: 5)
ATGGAGACCGACACACTGCTCCTGTGGGTCCTGCTCCTCTGGGTGCCAGG
AAGTACAGGAGATCACCATCACCATCACCACGACGACGACGACAAGCACC
CTCTTTCTCCCCAAGGCCATCCTGCCAGGTTACATCGCATAGTGCCCCGG
CTCCGAGATGTCTTTGGGTGGGGGAACCTCACCTGCCCAATCTGCAAAGG
TCTATTCACCGCCATCAACCTCGGGCTGAAGAAGGAACCCAATGTGGCTC
GCGTGGGCTCCGTGGCCATCAAGCTGTGCAATCTGCTGAAGATAGCACCA
CCTGCCGTGTGCCAATCCATTGTCCACCTCTTTGAGGATGACATGGTGGA
GGTGTGGAGACGCTCAGTGCTGAGCCCATCTGAGGCCTGTGGCCTGCTCC
TGGGCTCCACCTGTGGGCACTGGGACATTTTCTCATCTTGGAACATCTCT
TTGCCTACTGTGCCGAAGCCGCCCCCCAAACCCCCTAGCCCCCCAGCCCC
AGGTGCCCCTGTCAGCCGCATCCTCTTCCTCACTGACCTGCACTGGGATC
ATGACTACCTGGAGGGCACGGACCCTGACTGTGCAGACCCACTGTGCTGC
CGCCGGGGTTCTGGCCTGCCGCCCGCATCCCGGCCAGGTGCCGGATACTG
GGGCGAATACAGCAAGTGTGACCTGCCCCTGAGGACCCTGGAGAGCCTGT
TGAGTGGGCTGGGCCCAGCCGGCCCTTTTGATATGGTGTACTGGACAGGA
GACATCCCCGCACATGATGTCTGGCACCAGACTCGTCAGGACCAACTGCG
GGCCCTGACCACCGTCACAGCACTTGTGAGGAAGTTCCTGGGGCCAGTGC
CAGTGTACCCTGCTGTGGGTAACCATGAAAGCACACCTGTCAATAGCTTC
CCTCCCCCCTTCATTGAGGGCAACCACTCCTCCCGCTGGCTCTATGAAGC
GATGGCCAAGGCTTGGGAGCCCTGGCTGCCTGCCGAAGCCCTGCGCACCC
TCAGAATTGGGGGGTTCTATGCTCTTTCCCCATACCCCGGTCTCCGCCTC
ATCTCTCTCAATATGAATTTTTGTTCCCGTGAGAACTTCTGGCTCTTGAT
CAACTCCACGGATCCCGCAGGACAGCTCCAGTGGCTGGTGGGGGAGCTTC
AGGCTGCTGAGGATCGAGGAGACAAAGTGCATATAATTGGCCACATTCCC
CCAGGGCACTGTCTGAAGAGCTGGAGCTGGAATTATTACCGAATTGTAGC
CAGGTATGAGAACACCCTGGCTGCTCAGTTCTTTGGCCACACTCATGTGG
ATGAATTTGAGGTCTTCTATGATGAAGAGACTCTGAGCCGGCCGCTGGCT
GTAGCCTTCCTGGCACCCAGTGCAACTACCTACATCGGCCTTAATCCTGG
TTACCGTGTGTACCAAATAGATGGAAACTACTCCGGGAGCTCTCACGTGG
TCCTGGACCATGAGACCTACATCCTGAATCTGACCCAGGCAAACATACCG
GGAGCCATACCGCACTGGCAGCTTCTCTACAGGGCTCGAGAAACCTATGG
GCTGCCCAACACACTGCCTACCGCCTGGCACAACCTGGTATATCGCATGC
GGGGCGACATGCAACTTTTCCAGACCTTCTGGTTTCTCTACCATAAGGGC
CACCCACCCTCGGAGCCCTGTGGCACGCCCTGCCGTCTGGCTACTCTTTG
TGCCCAGCTCTCTGCCCGTGCTGACAGCCCTGCTCTGTGCCGCCACCTGA
TGCCAGATGGGAGCCTCCCAGAGGCCCAGAGCCTGTGGCCAAGGCCACTG
TTTTGCGGATTTCTGGGAGGCGGAGGAGGATCCGGAGGAGGAGGAAGCAA
TAACCAAAAGATTGTGAATATCAAAGAGAAAGTGGCTCAGATTGAGGCTG
GAGGCGGAGGAAGCGGCGGCGGAGGAAGCAATAATCAGAAAATCGTCAAC
ATTAAGGAAAAGGTCGCCCAGATTGAAGCAGGAGGCGGCGGCAGCGGCGG
AGGCGGAAGCAATAATCAGAAGATTGTTAACATCAAAGAAAAGGTGGCCC
AAATTGAAGCAGGAGGAGGAGGATCTGGAGGCGGAGGCAGCAATAACCAG
AAGATCGTCAACATCAAGGAAAAGGTGGCTCAGATCGAGGCAGGAGGCGG
AGGAAGCGGAGGGGGCGGCTCTAACAACCAGAAAATCGTGAACATCAAAG
AGAAAGTGGCTCAGATCGAAGCCTAG

Sequence 6. cDNA sequence of the expression cassette for human ASM control.

(SEQ ID NO: 6)
ATGGAGACCGACACACTGCTCCTGTGGGTCCTGCTCCTCTGGGTGCCAGG
AAGTACAGGAGATCACCATCACCATCACCACGACGACGACGACAAGCACC
CTCTTTCTCCCCAAGGCCATCCTGCCAGGTTACATCGCATAGTGCCCCGG
CTCCGAGATGTCTTTGGGTGGGGGAACCTCACCTGCCCAATCTGCAAAGG
TCTATTCACCGCCATCAACCTCGGGCTGAAGAAGGAACCCAATGTGGCTC
GCGTGGGCTCCGTGGCCATCAAGCTGTGCAATCTGCTGAAGATAGCACCA
CCTGCCGTGTGCCAATCCATTGTCCACCTCTTTGAGGATGACATGGTGGA
GGTGTGGAGACGCTCAGTGCTGAGCCCATCTGAGGCCTGTGGCCTGCTCC
TGGGCTCCACCTGTGGGCACTGGGACATTTTCTCATCTTGGAACATCTCT
TTGCCTACTGTGCCGAAGCCGCCCCCCAAACCCCCTAGCCCCCCAGCCCC
AGGTGCCCCTGTCAGCCGCATCCTCTTCCTCACTGACCTGCACTGGGATC
ATGACTACCTGGAGGGCACGGACCCTGACTGTGCAGACCCACTGTGCTGC
CGCCGGGGTTCTGGCCTGCCGCCCGCATCCCGGCCAGGTGCCGGATACTG
GGGCGAATACAGCAAGTGTGACCTGCCCCTGAGGACCCTGGAGAGCCTGT
TGAGTGGGCTGGGCCCAGCCGGCCCTTTTGATATGGTGTACTGGACAGGA
GACATCCCCGCACATGATGTCTGGCACCAGACTCGTCAGGACCAACTGCG
GGCCCTGACCACCGTCACAGCACTTGTGAGGAAGTTCCTGGGGCCAGTGC
CAGTGTACCCTGCTGTGGGTAACCATGAAAGCACACCTGTCAATAGCTTC
CCTCCCCCCTTCATTGAGGGCAACCACTCCTCCCGCTGGCTCTATGAAGC
GATGGCCAAGGCTTGGGAGCCCTGGCTGCCTGCCGAAGCCCTGCGCACCC
TCAGAATTGGGGGGTTCTATGCTCTTTCCCCATACCCCGGTCTCCGCCTC
ATCTCTCTCAATATGAATTTTTGTTCCCGTGAGAACTTCTGGCTCTTGAT
CAACTCCACGGATCCCGCAGGACAGCTCCAGTGGCTGGTGGGGGAGCTTC
AGGCTGCTGAGGATCGAGGAGACAAAGTGCATATAATTGGCCACATTCCC
CCAGGGCACTGTCTGAAGAGCTGGAGCTGGAATTATTACCGAATTGTAGC
CAGGTATGAGAACACCCTGGCTGCTCAGTTCTTTGGCCACACTCATGTGG
ATGAATTTGAGGTCTTCTATGATGAAGAGACTCTGAGCCGGCCGCTGGCT
GTAGCCTTCCTGGCACCCAGTGCAACTACCTACATCGGCCTTAATCCTGG
TTACCGTGTGTACCAAATAGATGGAAACTACTCCGGGAGCTCTCACGTGG
TCCTGGACCATGAGACCTACATCCTGAATCTGACCCAGGCAAACATACCG
GGAGCCATACCGCACTGGCAGCTTCTCTACAGGGCTCGAGAAACCTATGG
GCTGCCCAACACACTGCCTACCGCCTGGCACAACCTGGTATATCGCATGC
GGGGCGACATGCAACTTTTCCAGACCTTCTGGTTTCTCTACCATAAGGGC
CACCCACCCTCGGAGCCCTGTGGCACGCCCTGCCGTCTGGCTACTCTTTG
TGCCCAGCTCTCTGCCCGTGCTGACAGCCCTGCTCTGTGCCGCCACCTGA
TGCCAGATGGGAGCCTCCCAGAGGCCCAGAGCCTGTGGCCAAGGCCACTG
TTTTGCTAG

Sequence 7. cDNA sequence of the expression cassette for human alpha galactosidase (αGal) with one copy of the 2γ3 ICAM-1-targeting peptide at the amino terminus.

(SEQ ID NO: 7)
ATGGGCTGGTCCTGCATCATTCTGTTTCTGGTGGCTACCGCCACCGGCGT
GCACTCTGATCACCACCACCATCACCACGACGATGACGACAAGAACAACC
AGAAGATCGTCAACATCAAAGAGAAGGTCGCCCAGATCGAGGCTGGAGGC
GGAGGATCTGGTGGTGGCGGATCTGGATTCCTTGGCCTGGACAACGGCCT
GGCTAGAACCCCTACCATGGGATGGCTGCACTGGGAGAGATTCATGTGCA
ACCTGGACTGCCAAGAGGAACCCGACTCCTGCATCTCCGAGAAGCTGTTC
ATGGAAATGGCCGAGCTGATGGTGTCCGAAGGCTGGAAGGATGCCGGCTA
CGAGTACCTGTGCATCGACGACTGTTGGATGGCCCCTCAGAGAGACTCTG
AGGGCAGACTGCAGGCCGATCCTCAGAGATTTCCCCACGGCATCAGACAG
CTGGCCAACTACGTGCACTCCAAGGGCCTGAAGCTGGGCATCTATGCCGA
CGTGGGCAACAAGACCTGTGCCGGCTTTCCTGGCTCCTTCGGCTACTACG
ATATCGACGCCCAGACCTTCGCTGACTGGGGAGTCGATCTGCTGAAGTTC
GACGGCTGCTACTGCGACTCCCTGGAAAATCTGGCCGACGGCTACAAGCA
CATGTCTCTGGCCCTGAACCGGACCGGCAGATCCATCGTGTATAGCTGCG
AGTGGCCCCTGTACATGTGGCCCTTCCAGAAGCCTAACTACACCGAGATC
AGACAGTACTGCAACCACTGGCGGAACTTCGCCGACATCGACGATAGCTG
GAAGTCCATCAAGTCTATCCTGGACTGGACCTCCTTCAATCAAGAGCGGA
TCGTGGATGTGGCTGGCCCTGGCGGATGGAACGATCCTGATATGCTGGTC
ATCGGCAACTTCGGCCTGTCCTGGAACCAGCAAGTGACCCAGATGGCCCT
GTGGGCCATTATGGCCGCTCCTCTGTTCATGTCCAACGACCTGAGACACA
TCAGCCCTCAGGCCAAGGCTCTGCTGCAGGACAAGGATGTGATCGCTATC
AACCAGGATCCTCTGGGCAAGCAGGGCTACCAGTTGAGACAGGGCGACAA
CTTTGAAGTGTGGGAAAGACCCCTGTCCGGCCTGGCATGGGCTGTCGCCA
TGATCAACAGACAAGAGATCGGCGGACCCCGGTCCTACACAATCGCTGTT
GCTTCTCTCGGCAAAGGCGTGGCCTGCAATCCTGCCTGTTTCATCACACA
GCTGCTGCCCGTGAAGAGAAAGCTGGGCTTTTACGAGTGGACCTCTCGGC
TGCGGTCCCACATCAATCCTACCGGAACAGTGCTGCTGCAGCTGGAAAAC
ACCATGCAGATGTCCCTGAAGGACCTGCTGTGA

Sequence 8. cDNA sequence of the expression cassette for human αGal with five tandem-repeats of the 2γ3 ICAM-1-targeting peptide at the carboxyl terminus.

(SEQ ID NO: 8)
ATGGGCTGGTCCTGCATCATTCTGTTTCTGGTGGCTACCGCCACCGGCGT
GCACTCTGATCACCACCACCATCACCACGACGATGACGACAAGCTGGACA
ACGGCCTGGCTAGAACCCCTACCATGGGATGGCTGCACTGGGAGAGATTC
ATGTGCAACCTGGACTGCCAAGAGGAACCCGACTCCTGCATCTCCGAGAA
GCTGTTCATGGAAATGGCCGAGCTGATGGTGTCCGAAGGCTGGAAGGATG
CCGGCTACGAGTACCTGTGCATCGACGACTGTTGGATGGCCCCTCAGAGA
GACTCTGAGGGCAGACTGCAGGCCGATCCTCAGAGATTTCCCCACGGCAT
CAGACAGCTGGCCAACTACGTGCACTCCAAGGGCCTGAAGCTGGGCATCT
ATGCCGACGTGGGCAACAAGACCTGTGCCGGCTTTCCTGGCTCCTTCGGC
TACTACGATATCGACGCCCAGACCTTCGCTGACTGGGGAGTCGATCTGCT
GAAGTTCGACGGCTGCTACTGCGACTCCCTGGAAAATCTGGCCGACGGCT
ACAAGCACATGTCTCTGGCCCTGAACCGGACCGGCAGATCCATCGTGTAT
AGCTGCGAGTGGCCCCTGTACATGTGGCCCTTCCAGAAGCCTAACTACAC
CGAGATCAGACAGTACTGCAACCACTGGCGGAACTTCGCCGACATCGACG
ATAGCTGGAAGTCCATCAAGTCTATCCTGGACTGGACCTCCTTCAATCAA
GAGCGGATCGTGGATGTGGCTGGCCCTGGCGGATGGAACGATCCTGATAT
GCTGGTCATCGGCAACTTCGGCCTGTCCTGGAACCAGCAAGTGACCCAGA
TGGCCCTGTGGGCCATTATGGCCGCTCCTCTGTTCATGTCCAACGACCTG
AGACACATCAGCCCTCAGGCCAAGGCTCTGCTGCAGGACAAGGATGTGAT
CGCTATCAACCAGGATCCTCTGGGCAAGCAGGGCTACCAGTTGAGACAGG
GCGACAACTTTGAAGTGTGGGAAAGACCCCTGTCCGGCCTGGCATGGGCT
GTCGCCATGATCAACAGACAAGAGATCGGCGGACCCCGGTCCTACACAAT
CGCTGTTGCTTCTCTCGGCAAAGGCGTGGCCTGCAATCCTGCCTGTTTCA
TCACACAGCTGCTGCCCGTGAAGAGAAAGCTGGGCTTTTACGAGTGGACC
TCTCGGCTGCGGTCCCACATCAATCCTACCGGAACAGTGCTGCTGCAGCT
GGAAAACACCATGCAGATGTCCCTGAAGGACCTGCTGGGATTCCTTGGCG
GAGGCGGAGGATCTGGTGGTGGCGGATCTAACAACCAGAAGATCGTCAAC
ATCAAAGAGAAGGTCGCCCAGATCGAGGCTGGCGGCGGTGGATCAGGTGG
CGGAGGAAGCAACAATCAGAAAATTGTGAATATCAAAGAAAAAGTGGCTC
AGATTGAAGCAGGCGGCGGAGGTAGCGGAGGTGGTGGCTCTAACAATCAA
AAAATCGTTAACATCAAAGAGAAAGTTGCTCAAATCGAAGCCGGCGGTGG
TGGTTCTGGCGGTGGTGGTAGTAACAATCAAAAGATCGTCAATATCAAAG
AAAAGGTGGCACAAATCGAGGCAGGCGGAGGCGGCTCTGGCGGCGGAGGA
TCAAACAATCAGAAGATCGTTAACATCAAAGAAAAAGTGGCCCAAATTGA
GGCCTGA

Sequence 9. cDNA sequence of the expression cassette for human αGal control.

(SEQ ID NO: 9)
ATGGGCTGGTCCTGCATCATTCTGTTTCTGGTGGCTACCGCCACCGGCGT
GCACTCTGATCACCACCACCATCACCACGACGATGACGACAAGCTGGACA
ACGGCCTGGCTAGAACCCCTACCATGGGATGGCTGCACTGGGAGAGATTC
ATGTGCAACCTGGACTGCCAAGAGGAACCCGACTCCTGCATCTCCGAGAA
GCTGTTCATGGAAATGGCCGAGCTGATGGTGTCCGAAGGCTGGAAGGATG
CCGGCTACGAGTACCTGTGCATCGACGACTGTTGGATGGCCCCTCAGAGA
GACTCTGAGGGCAGACTGCAGGCCGATCCTCAGAGATTTCCCCACGGCAT
CAGACAGCTGGCCAACTACGTGCACTCCAAGGGCCTGAAGCTGGGCATCT
ATGCCGACGTGGGCAACAAGACCTGTGCCGGCTTTCCTGGCTCCTTCGGC
TACTACGATATCGACGCCCAGACCTTCGCTGACTGGGGAGTCGATCTGCT
GAAGTTCGACGGCTGCTACTGCGACTCCCTGGAAAATCTGGCCGACGGCT
ACAAGCACATGTCTCTGGCCCTGAACCGGACCGGCAGATCCATCGTGTAT
AGCTGCGAGTGGCCCCTGTACATGTGGCCCTTCCAGAAGCCTAACTACAC
CGAGATCAGACAGTACTGCAACCACTGGCGGAACTTCGCCGACATCGACG
ATAGCTGGAAGTCCATCAAGTCTATCCTGGACTGGACCTCCTTCAATCAA
GAGCGGATCGTGGATGTGGCTGGCCCTGGCGGATGGAACGATCCTGATAT
GCTGGTCATCGGCAACTTCGGCCTGTCCTGGAACCAGCAAGTGACCCAGA
TGGCCCTGTGGGCCATTATGGCCGCTCCTCTGTTCATGTCCAACGACCTG
AGACACATCAGCCCTCAGGCCAAGGCTCTGCTGCAGGACAAGGATGTGAT
CGCTATCAACCAGGATCCTCTGGGCAAGCAGGGCTACCAGTTGAGACAGG
GCGACAACTTTGAAGTGTGGGAAAGACCCCTGTCCGGCCTGGCATGGGCT
GTCGCCATGATCAACAGACAAGAGATCGGCGGACCCCGGTCCTACACAAT
CGCTGTTGCTTCTCTCGGCAAAGGCGTGGCCTGCAATCCTGCCTGTTTCA
TCACACAGCTGCTGCCCGTGAAGAGAAAGCTGGGCTTTTACGAGTGGACC
TCTCGGCTGCGGTCCCACATCAATCCTACCGGAACAGTGCTGCTGCAGCT
GGAAAACACCATGCAGATGICCCTGAAGGACCTGCTGTGA

Sequence 10. cDNA sequence of the expression cassette for human glucocerebrosidase (GCasc) with one copy of the 2γ3 ICAM-1-targeting peptide at the amino terminus.

(SEQ ID NO: 10)
ATGGGCTGGTCCTGCATCATTCTGTTTCTGGTGGCTACCGCCACCGGCGT
GCACTCTGATCACCACCACCATCACCACGACGATGACGACAAGAACAACC
AGAAGATCGTCAACATCAAAGAGAAGGTCGCCCAGATCGAGGCTGGCGGC
GGAGGATCTGGCGGAGGCGGATCTGGATTTTTGGGAGCCAGACCTTGCAT
CCCCAAGTCCTTCGGCTACTCCTCTGTCGTGTGCGTGTGCAACGCCACCT
ACTGCGACAGCTTCGACCCTCCTACCTTTCCTGCTCTGGGCACATTCTCC
AGATACGAGTCCACCAGATCCGGCAGACGGATGGAACTGAGCATGGGCCC
TATCCAGGCTAACCATACCGGCACAGGACTGCTGCTGACACTGCAGCCCG
AGCAGAAATTCCAGAAAGTGAAAGGCTTCGGCGGAGCCATGACCGATGCC
GCCGCTCTGAATATTCTGGCTCTGAGCCCTCCTGCTCAGAACCTGCTGCT
CAAGTCCTACTTCTCCGAGGAAGGCATCGGCTACAACATCATCCGGGTGC
CAATGGCCTCCTGCGACTTCTCTATCCGGACCTACACCTACGCTGACACC
CCTGACGATTTCCAGCTGCACAACTTCAGCCTGCCTGAAGAGGACACCAA
GCTGAAGATCCCTCTGATCCACAGAGCCCTGCAGCTGGCTCAGAGGCCTG
TTTCTCTGCTGGCCTCTCCTTGGACCTCTCCAACCTGGCTGAAAACAAAT
GGCGCCGTGAACGGCAAGGGCTCCCTGAAAGGACAACCCGGCGATATCTA
CCACCAGACCTGGGCCAGATACTTCGTGAAGTTCCTGGACGCCTACGCCG
AGCACAAGCTGCAGTTTTGGGCTGTGACCGCCGAGAACGAGCCTTCTGCT
GGACTGCTGTCTGGCTACCCTTTCCAGTGCCTGGGCTTTACCCCTGAGCA
CCAGAGAGACTTTATCGCCAGAGATCTGGGCCCCACACTGGCCAATTCTA
CCCACCATAATGTGCGGCTGCTGATGCTGGACGACCAGAGACTGCTGTTG
CCCCACTGGGCTAAAGTGGTGCTGACCGATCCTGAGGCCGCCAAATACGT
GCACGGAATCGCCGTGCACTGGTATCTGGACTTTCTGGCCCCTGCCAAGG
CTACCCTGGGCGAGACACATAGACTGTTCCCCAACACCATGCTGTTCGCC
TCTGAGGCCTGTGTGGGCTCCAAGTTCTGGGAGCAGTCTGTGCGACTCGG
CTCTTGGGATAGAGGCATGCAGTACTCCCACTCCATCATCACCAACCTGC
TGTACCACGTCGTCGGCTGGACCGATTGGAACCTGGCACTGAATCCTGAA
GGCGGCCCTAACTGGGTCCGAAACTTCGTGGACTCCCCTATCATCGTGGA
CATCACCAAGGACACCTTCTACAAGCAGCCCATGTTCTACCATCTGGGCC
ACTTCAGCAAGTTCATCCCCGAGGGCTCTCAGAGAGTCGGCCTGGTTGCC
TCTCAGAAGAACGACCTGGACGCTGTGGCTCTGATGCACCCTGATGGATC
TGCTGTGGTGGTCGTGCTGAACCGGTCCTCCAAAGATGTGCCCCTGACCA
TCAAGGATCCCGCCGTGGGATTCCTGGAAACCATCTCTCCTGGCTACTCC
ATCCACACCTACCTGTGGCGTAGACAGTGA

Sequence 11. cDNA sequence of the expression cassette for human GCase with five tandem-repeats of the 2γ3 ICAM-1-targeting peptide at the amino terminus.

(SEQ ID NO: 11)
ATGGGCTGGTCCTGCATCATTCTGTTTCTGGTGGCTACCGCCACCGGCGTGCACTCTGATCA
CCACCACCATCACCACGACGATGACGACAAGAACAACCAGAAGATCGTCAACATCAAAGAGA
AGGTCGCCCAGATCGAGGCTGGCGGCGGAGGATCTGGCGGAGGCGGATCTAACAATCAGAAA
ATTGTGAATATCAAAGAAAAAGTGGCTCAGATTGAAGCCGGCGGTGGTGGTAGCGGTGGCGG
AGGAAGTAACAATCAAAAGATCGTGAACATCAAAGAAAAAGTTGCACAAATCGAGGCAGGCG
GTGGCGGCAGCGGAGGTGGTGGATCCAACAACCAGAAAATCGTGAACATCAAAGAAAAGGTG
GCCCAAATCGAAGCCGGCGGAGGCGGTTCAGGCGGCGGAGGTTCAAACAATCAGAAGATCGT
TAATATCAAAGAAAAGGTTGCCCAGATTGAGGCAGGCGGAGGTGGAAGCGGCGGAGGCGGCT
CTGGATTTTTGGGAGCCAGACCTTGCATCCCCAAGTCCTTCGGCTACTCCTCTGTCGTGTGC
GTGTGCAACGCCACCTACTGCGACAGCTTCGACCCTCCTACCTTTCCTGCTCTGGGCACATT
CTCCAGATACGAGTCCACCAGATCCGGCAGACGGATGGAACTGAGCATGGGCCCTATCCAGG
CTAACCATACCGGCACAGGACTGCTGCTGACACTGCAGCCCGAGCAGAAATTCCAGAAAGTG
AAAGGCTTCGGCGGAGCCATGACCGATGCCGCCGCTCTGAATATTCTGGCTCTGAGCCCTCC
TGCTCAGAACCTGCTGCTCAAGTCCTACTTCTCCGAGGAAGGCATCGGCTACAACATCATCC
GGGTGCCAATGGCCTCCTGCGACTTCTCTATCCGGACCTACACCTACGCTGACACCCCTGAC
GATTTCCAGCTGCACAACTTCAGCCTGCCTGAAGAGGACACCAAGCTGAAGATCCCTCTGAT
CCACAGAGCCCTGCAGCTGGCTCAGAGGCCTGTTTCTCTGCTGGCCTCTCCTTGGACCTCTC
CAACCTGGCTGAAAACAAATGGCGCCGTGAACGGCAAGGGCTCCCTGAAAGGACAACCCGGC
GATATCTACCACCAGACCTGGGCCAGATACTTCGTGAAGTTCCTGGACGCCTACGCCGAGCA
CAAGCTGCAGTTTTGGGCTGTGACCGCCGAGAACGAGCCTTCTGCTGGACTGCTGTCTGGCT
ACCCTTTCCAGTGCCTGGGCTTTACCCCTGAGCACCAGAGAGACTTTATCGCCAGAGATCTG
GGCCCCACACTGGCCAATTCTACCCACCATAATGTGCGGCTGCTGATGCTGGACGACCAGAG
ACTGCTGTTGCCCCACTGGGCTAAAGTGGTGCTGACCGATCCTGAGGCCGCCAAATACGTGC
ACGGAATCGCCGTGCACTGGTATCTGGACTTTCTGGCCCCTGCCAAGGCTACCCTGGGCGAG
ACACATAGACTGTTCCCCAACACCATGCTGTTCGCCTCTGAGGCCTGTGTGGGCTCCAAGTT
CTGGGAGCAGTCTGTGCGACTCGGCTCTTGGGATAGAGGCATGCAGTACTCCCACTCCATCA
TCACCAACCTGCTGTACCACGTCGTCGGCTGGACCGATTGGAACCTGGCACTGAATCCTGAA
GGCGGCCCTAACTGGGTCCGAAACTTCGTGGACTCCCCTATCATCGTGGACATCACCAAGGA
CACCTTCTACAAGCAGCCCATGTTCTACCATCTGGGCCACTTCAGCAAGTTCATCCCCGAGG
GCTCTCAGAGAGTCGGCCTGGTTGCCTCTCAGAAGAACGACCTGGACGCTGTGGCTCTGATG
CACCCTGATGGATCTGCTGTGGTGGTCGTGCTGAACCGGTCCTCCAAAGATGTGCCCCTGAC
CATCAAGGATCCCGCCGTGGGATTCCTGGAAACCATCTCTCCTGGCTACTCCATCCACACCT
ACCTGTGGCGTAGACAGTGA

Sequence 12. cDNA sequence of the expression cassette for human GCase control.

(SEQ ID NO: 12)
ATGGGCTGGTCCTGCATCATTCTGTTTCTGGTGGCTACCGCCACCGGCGTGCACTCTGATCA
CCACCACCATCACCACGACGATGACGACAAGCTGGACAACGGCCTGGCTAGAACCCCTACCA
TGGGATGGCTGCACTGGGAGAGATTCATGTGCAACCTGGACTGCCAAGAGGAACCCGACTCC
TGCATCTCCGAGAAGCTGTTCATGGAAATGGCCGAGCTGATGGTGTCCGAAGGCTGGAAGGA
TGCCGGCTACGAGTACCTGTGCATCGACGACTGTTGGATGGCCCCTCAGAGAGACTCTGAGG
GCAGACTGCAGGCCGATCCTCAGAGATTTCCCCACGGCATCAGACAGCTGGCCAACTACGTG
CACTCCAAGGGCCTGAAGCTGGGCATCTATGCCGACGTGGGCAACAAGACCTGTGCCGGCTT
TCCTGGCTCCTTCGGCTACTACGATATCGACGCCCAGACCTTCGCTGACTGGGGAGTCGATC
TGCTGAAGTTCGACGGCTGCTACTGCGACTCCCTGGAAAATCTGGCCGACGGCTACAAGCAC
ATGTCTCTGGCCCTGAACCGGACCGGCAGATCCATCGTGTATAGCTGCGAGTGGCCCCTGTA
CATGTGGCCCTTCCAGAAGCCTAACTACACCGAGATCAGACAGTACTGCAACCACTGGCGGA
ACTTCGCCGACATCGACGATAGCTGGAAGTCCATCAAGTCTATCCTGGACTGGACCTCCTTC
AATCAAGAGCGGATCGTGGATGTGGCTGGCCCTGGCGGATGGAACGATCCTGATATGCTGGT
CATCGGCAACTTCGGCCTGTCCTGGAACCAGCAAGTGACCCAGATGGCCCTGTGGGCCATTA
TGGCCGCTCCTCTGTTCATGTCCAACGACCTGAGACACATCAGCCCTCAGGCCAAGGCTCTG
CTGCAGGACAAGGATGTGATCGCTATCAACCAGGATCCTCTGGGCAAGCAGGGCTACCAGTT
GAGACAGGGCGACAACTTTGAAGTGTGGGAAAGACCCCTGTCCGGCCTGGCATGGGCTGTCG
CCATGATCAACAGACAAGAGATCGGCGGACCCCGGTCCTACACAATCGCTGTTGCTTCTCTC
GGCAAAGGCGTGGCCTGCAATCCTGCCTGTTTCATCACACAGCTGCTGCCCGTGAAGAGAAA
GCTGGGCTTTTACGAGTGGACCTCTCGGCTGCGGTCCCACATCATCCTACCGGAACAGTGC
TGCTGCAGCTGGAAAACACCATGCAGATGTCCCTGAAGGACCTGCTGTGA

Sequence 13. Amino acid sequence of the expression cassette for human acid sphingomyelinase (ASM) with one copy of the 2γ3 ICAM-1-targeting peptide at the amino terminus

(SEQ ID NO: 13)
METDTLLLWVLLLWVPGSTGDHHHHHHDDDDKNNQKIVNIKEKVAQIEAG
GGGSGGGGSGFLGHPLSPQGHPARLHRIVPRLRDVFGWGNLTCPICKGLF
TAINLGLKKEPNVARVGSVAIKLCNLLKIAPPAVCQSIVHLFEDDMVEVW
RRSVLSPSEACGLLLGSTCGHWDIFSSWNISLPTVPKPPPKPPSPPAPGA
PVSRILFLTDLHWDHDYLEGTDPDCADPLCCRRGSGLPPASRPGAGYWGE
YSKCDLPLRTLESLLSGLGPAGPFDMVYWTGDIPAHDVWHQTRQDQLRAL
TTVTALVRKFLGPVPVYPAVGNHESTPVNSFPPPFIEGNHSSRWLYEAMA
KAWEPWLPAEALRTLRIGGFYALSPYPGLRLISLNMNFCSRENFWLLINS
TDPAGQLQWLVGELQAAEDRGDKVHIIGHIPPGHCLKSWSWNYYRIVARY
ENTLAAQFFGHTHVDEFEVFYDEETLSRPLAVAFLAPSATTYIGLNPGYR
VYQIDGNYSGSSHVVLDHETYILNLTQANIPGAIPHWQLLYRARETYGLP
NTLPTAWHNLVYRMRGDMQLFQTFWFLYHKGHPPSEPCGTPCRLATLCAQ
LSARADSPALCRHLMPDGSLPEAQSLWPRPLFC*

Sequence 14. Amino acid sequence of the expression cassette for human ASM with five tandem-repeats of the 2γ3 ICAM-1-targeting peptide at the amino terminus.

(SEQ ID NO: 14)
METDTLLLWVLLLWVPGSTGDHHHHHHDDDDKNNQKIVNIKEKVAQIEAG
GGGSGGGGSNNQKIVNIKEKVAQIEAGGGGSGGGGSNNQKIVNIKEKVAQ
IEAGGGGSGGGGSNNQKIVNIKEKVAQIEAGGGGSGGGGSNNQKIVNIKE
KVAQIEAGGGGSGGGGSGFLGHPLSPQGHPARLHRIVPRLRDVFGWGNLT
CPICKGLFTAINLGLKKEPNVARVGSVAIKLCNLLKIAPPAVCQSIVHLF
EDDMVEVWRRSVLSPSEACGLLLGSTCGHWDIFSSWNISLPTVPKPPPKP
PSPPAPGAPVSRILFLTDLHWDHDYLEGTDPDCADPLCCRRGSGLPPASR
PGAGYWGEYSKCDLPLRTLESLLSGLGPAGPFDMVYWTGDIPAHDVWHQT
RQDQLRALTTVTALVRKFLGPVPVYPAVGNHESTPVNSFPPPFIEGNHSS
RWLYEAMAKAWEPWLPAEALRTLRIGGFYALSPYPGLRLISLNMNFCSRE
NFWLLINSTDPAGQLQWLVGELQAAEDRGDKVHIIGHIPPGHCLKSWSWN
YYRIVARYENTLAAQFFGHTHVDEFEVFYDEETLSRPLAVAFLAPSATTY
IGLNPGYRVYQIDGNYSGSSHVVLDHETYILNLTQANIPGAIPHWQLLYR
ARETYGLPNTLPTAWHNLVYRMRGDMQLFQTFWFLYHKGHPPSEPCGTPC
RLATLCAQLSARADSPALCRHLMPDGSLPEAQSLWPRPLFC*

Sequence 15. Amino acid sequence of the expression cassette for human ASM with ten tandem-repeats of the 2γ3 ICAM-1-targeting peptide at the amino terminus.

(SEQ ID NO: 15)
METDTLLLWVLLLWVPGSTGDHHHHHHDDDDKNNQKIVNIKEKVAQIEAG
GGGSGGGGSNNQKIVNIKEKVAQIEAGGGGSGGGGSNNQKIVNIKEKVAQ
IEAGGGGSGGGGSNNQKIVNIKEKVAQIEAGGGGSGGGGSNNQKIVNIKE
KVAQIEAGGGGSGGGGSNNQKIVNIKEKVAQIEAGGGGSGGGGSNNQKIV
NIKEKVAQIEAGGGGSGGGGSNNQKIVNIKEKVAQIEAGGGGSGGGGSNN
KQIVNIKEKVAQIEAGGGGSGGGGSNNQKIVNIKEKVAQIEAGGGGSGGG
GSFLGHPLSPQGHPARLHRIVPRLRDVFGWGNLTCPICKGLFTAINLGLK
KEPNVARVGSVAIKLCNLLKIAPPAVCQSIVHLFEDDMVEVWRRSVLSPS
EACGLLLGSTCGHWDIFSSWNISLPTVPKPPPKPPSPPAPGAPVSRILFL
TDLHWDHDYLEGTDPDCADPLCCRRGSGLPPASRPGAGYWGEYSKCDLPL
PLRTLESLLSGLGPAGPFDMVYWTGDIPAHDVWHQTRQDQLRALTTVTAL
VRKFLGPVPVYPAVGNHESTPVNSFPPPFIEGNHSSRWLYEAMAKAWEPW
LPAEALRTLRIGGFYALSPYPGLRLISLNMNFCSRENFWLLINSTDPAGQ
LQWLVGELQAAEDRGDKVHIIGHIPPGHCLKSWSWNYYRIVARYENTLAA
QFFGHTHVDEFEVFYDEETLSRPLAVAFLAPSATTYIGLNPGYRVYQIDG
NYSGSSHVVLDHETYILNLTQANIPGAIPHWQLLYRARETYGLPNTLPTA
WHNLVYRMRGDMQLFQTFWFLYHKGHPPSEPCGTPCRLATLCAQLSARAD
SPALCHRLMPDGSLPEAQSLWPRPLFC*

Sequence 16. Amino acid sequence of the expression cassette for human ASM with five tandem-repeats of the 2γ3 ICAM-1-targeting peptide at the carboxyl terminus.

(SEQ ID NO: 16)
METDTLLLWVLLLWVPGSTGDHHHHHHDDDDKHPLSPQGHPARLHRIVPR
LRDVFGWGNLTCPICKGLFTAINLGLKKEPNVARVGSVAIKLCNLLKIAP
PAVCQSIVHLFEDDMVEVWRRSVLSPSEACGLLLGSTCGHWDIFSSWNIS
LPTVPKPPPKPPSPPAPGAPVSRILFLTDLHWDHDYLEGTDPDCADPLCC
RRGSGLPPASRPGAGYWGEYSKCDLPLRTLESLLSGLGPAGPFDMVYWTG
DIPAHDVWHQTRQDQLRALTTVTALVRKFLGPVPVYPAVGNHESTPVNSF
PPPFIEGNHSSRWLYEAMAKAWEPWLPAEALRTLRIGGFYALSPYPGLRL
ISLNMNFCSRENFWLLINSTDPAGQLQWLVGELQAAEDRGDKVHIIGHIP
PGHCLKSWSWNYYRIVARYENTLAAQFFGHTHVDEFEVFYDEETLSRPLA
VAFLAPSATTYIGLNPGYRVYQIDGNYSGSSHVVLDHETYILNLTQANIP
GAIPHWQLLYRARETYGLPNTLPTAWHNLVYRMRGDMQLFQTFWFLYHKG
HPPSEPCGTPCRLATLCAQLSARADSPALCRHLMPDGSLPEAQSLWPRPL
FCGFLGGGGGSGGGGSNNQKIVNIKEKVAQIEAGGGGSGGGGSNNQKIVN
IKEKVAQIEAGGGGSGGGGSNNQKIVNIKEKVAQIEAGGGGSGGGGSNNQ
KIVNIKEKVAQIEAGGGGSGGGGSNNQKIVNIKEKVAQIEA*

Sequence 17. Amino acid sequence of the expression cassette for human ASM control.

(SEQ ID NO: 17)
METDTLLLWVLLLWVPGSTGDHHHHHHDDDDKHPLSPQGHPARLHRIVPR
LRDVFGWGNLTCPICKGLFTAINLGLKKEPNVARVGSVAIKLCNLLKIAP
PAVCQSIVHLFEDDMVEVWRRSVLSPSEACGLLLGSTCGHWDIFSSWNIS
LPTVPKPPPKPPSPPAPGAPVSRILFLTDLHWDHDYLEGTDPDCADPLCC
RRGSGLPPASRPGAGYWGEYSKCDLPLRTLESLLSGLGPAGPFDMVYWTG
DIPAHDVWHQTRQDQLRALTTVTALVRKFLGPVPVYPAVGNHESTPVNSF
PPPFIEGNHSSRWLYEAMAKAWEPWLPAEALRTLRIGGFYALSPYPGLRL
ISLNMNFCSRENFWLLINSTDPAGQLQWLVGELQAAEDRGDKVHIIGHIP
PGHCLKSWSWNYYRIVARYENTLAAQFFGHTHVDEFEVFYDEETLSRPLA
VAFLAPSATTYIGLNPGYRVYQIDGNYSGSSHVVLDHETYILNLTQANIP
GAIPHWQLLYRARETYGLPNTLPTAWHNLVYRMRGDMQLFQTFWFLYHKG
HPPSEPCGTPCRLATLCAQLSARADSPALCRHLMPDGSLPEAQSLWPRPL
FC*

Sequence 18. Amino acid sequence of the expression cassette for human alpha galactosidase (αGal) with one copy of the 2γ3 ICAM-1-targeting peptide at the amino terminus.

(SEQ ID NO: 18)
MGWSCIILFLVATATGVHSDHHHHHHDDDDKNNQKIVNIKEKVAQIEAGG
GGSGGGGSGFLGLDNGLARTPTMGWLHWERFMCNLDCQEEPDSCISEKLF
MEMAELMVSEGWKDAGYEYLCIDDCWMAPQRDSEGRLQADPQRFPHGIRQ
LANYVHSKGLKLGIYADVGNKTCAGFPGSFGYYDIDAQTFADWGVDLLKF
DGCYCDSLENLADGYKHMSLALNRTGRSIVYSCEWPLYMWPFQKPNYTEI
RQYCNHWRNFADIDDSWKSIKSILDWTSFNQERIVDVAGPGGWNDPDMLV
IGNFGLSWNQQVTQMALWAIMAAPLFMSNDLRHISPQAKALLQDKDVIAI
NQDPLGKQGYQLRQGDNFEVWERPLSGLAWAVAMINRQEIGGPRSYTIAV
ASLGKGVACNPACFITQLLPVKRKLGFYEWTSRLRSHINPTGTVLLQLEN
TMQMSLKDLL*

Sequence 19. Amino acid sequence of the expression cassette for human αGal with five tandem-repeats of the 2γ3 ICAM-1-targeting peptide at the carboxyl terminus.

(SEQ ID NO: 19)
MGWSCIILFLVATATGVHSDHHHHHHDDDDKLDNGLARTPTMGWLHWERF
MCNLDCQEEPDSCISEKLFMEMAELMVSEGWKDAGYEYLCIDDCWMAPQR
DSEGRLQADPQRFPHGIRQLANYVHSKGLKLGIYADVGNKTCAGFPGSFG
MSYYDIDAQTFADWGVDLLKFDGCYCDSLENLADGYKHLALNRTGRSIVY
SCEWPLYMWPFQKPNYTEIRQYCNHWRNFADIDDSWKSIKSILDWTSFNQ
ERIVDVAGPGGWNDPDMLVIGNFGLSWNQQVTQMALWAIMAAPLFMSNDL
RHISPQAKALLQDKDVIAINQDPLGKQGYQLRQGDNFEVWERPLSGLAWA
VAMINRQEIGGPRSYTIAVASLGKGVACNPACFITQLLPVKRKLGFYEWT
SRLRSHINPTGTVLLQLENTMQMSLKDLLGFLGGGGGSGGGGSNNQKIVN
IKEKVAQIEAGGGGSGGGGSNNQKIVNIKEKVAQIEAGGGGSGGGGSNNQ
KIVNIKEKVAQIEAGGGGSGGGGSNNQKIVNIKEKVAQIEAGGGGSGGGG
SNNQKIVNIKEKVAQIEA*

Sequence 20. Amino acid sequence of the expression cassette for human αGal control.

(SEQ ID NO: 20)
MGWSCIILFLVATATGVHSDHHHHHHDDDDKLDNGLARTPTMGWLHWERF
MCNLDCQEEPDSCISEKLFMEMAELMVSEGWKDAGYEYLCIDDCWMAPQR
DSEGRLQADPQRFPHGIRQLANYVHSKGLKLGIYADVGNKTCAGFPGSFG
YYDIDAQTFADWGVDLLKFDGCYCDSLENLADGYKHMSLALNRTGRSIVY
SCEWPLYMWPFQKPNYTEIRQYCNHWRNFADIDDSWKSIKSILDWTSFNQ
ERIVDVAGPGGWNDPDMLVIGNFGLSWNQQVTQMALWAIMAAPLFMSNDL
RHISPQAKALLQDKDVIAINQDPLGKQGYQLRQGDNFEVWERPLSGLAWA
VAMINRQEIGGPRSYTIAVASLGKGVACNPACFITQLLPVKRKLGFYEWT
SRLRSHINPTGTVLLQLENTMQMSLKDLL*

Sequence 21. Amino acid sequence of the expression cassette for human glucocerebrosidase (GCase) with one copy of the 2γ3 ICAM-1-targeting peptide at the amino terminus.

(SEQ ID NO: 21)
MGWSCIILFLVATATGVHSDHHHHHHDDDDKNNQKIVNIKEKVAQIEAGG
GGSGGGGSGFLGARPCIPKSFGYSSVVCVCNATYCDSFDPPTFPALGTFS
RYESTRSGRRMELSMGPIQANHTGTGLLLTLQPEQKFQKVKGFGGAMTDA
AALNILALSPPAQNLLLKSYFSEEGIGYNIIRVPMASCDFSIRTYTYADT
PDDFQLHNFSLPEEDTKLKIPLIHRALQLAQRPVSLLASPWTSPTWLKTN
GAVNGKGSLKGQPGDIYHQTWARYFVKFLDAYAEHKLQFWAVTAENEPSA
GLLSGYPFQCLGFTPEHQRDFIARDLGPTLANSTHHNVRLLMLDDQRLLL
PHWAKVVLTDPEAAKYVHGIAVHWYLDFLAPAKATLGETHRLFPNTMLFA
SEACVGSKFWEQSVRLGSWDRGMQYSHSIITNLLYHVVGWTDWNLALNPE
GGPNWVRNFVDSPIIVDITKDTFYKQPMFYHLGHFSKFIPEGSQRVGLVA
SQKNDLDAVALMHPDGSAVVVVLNRSSKDVPLTIKDPAVGFLETISPGYS
IHTYLWRRQ*

Sequence 22. Amino acid sequence of the expression cassette for human GCase with five tandem-repeats of the 2γ3 ICAM-1-targeting peptide at the amino terminus.

(SEQ ID NO: 22)
MGWSCIILFLVATATGVHSDHHHHHHDDDDKNNQKIVNIKEKVAQIEAGG
GGSGGGGSNNQKIVNIKEKVAQIEAGGGGSGGGGSNNQKIVNIKEKVAQI
EAGGGGSGGGGSNNQKIVNIKEKVAQIEAGGGGSGGGGSNNQKIVNIKEK
VAQIEAGGGGSGGGGSGFLGARPCIPKSFGYSSVVCVCNATYCDSFDPPT
FPALGTFSRYESTRSGRRMELSMGPIQANHTGTGLLLTLQPEQKFQKVKG
FGGAMTDAAALNILALSPPAQNLLLKSYFSEEGIGYNIIRVPMASCDFSI
RTYTYADTPDDFQLHNFSLPEEDTKLKIPLIHRALQLAQRPVSLLASPWT
SPTWLKTNGAVNGKGSLKGQPGDIYHQTWARYFVKFLDAYAEHKLQFWAV
TAENEPSAGLLSGYPFQCLGFTPEHQRDFIARDLGPTLANSTHHNVRLLM
LDDQRLLLPHWAKVVLTDPEAAKYVHGIAVHWYLDFLAPAKATLGETHRL
FPNTMLFASEACVGSKFWEQSVRLGSWDRGMQYSHSIITNLLYHVVGWTD
WNLALNPEGGPNWVRNFVDSPIIVDITKDTFYKQPMFYHLGHFSKFIPEG
SQRVGLVASQKNDLDAVALMHPDGSAVVVVLNRSSKDVPLTIKDPAVGFL
ETISPGYSIHTYLWRRQ*

Sequence 23. Amino acid sequence of the expression cassette for human GCase control.

(SEQ ID NO: 23)
MGWSCIILFLVATATGVHSDHHHHHHDDDDKARPCIPKSFGYSSVVCVCN
ATYCDSFDPPTFPALGTFSRYESTRSGRRMELSMGPIQANHTGTGLLLTL
QPEQKFQKVKGFGGAMTDAAALNILALSPPAQNLLLKSYFSEEGIGYNII
RVPMASCDFSIRTYTYADTPDDFQLHNFSLPEEDTKLKIPLIHRALQLAQ
RPVSLLASPWTSPTWLKTNGAVNGKGSLKGQPGDIYHQTWARYFVKFLDA
YAEHKLQFWAVTAENEPSAGLLSGYPFQCLGFTPEHQRDFIARDLGPTLA
NSTHHNVRLLMLDDQRLLLPHWAKVVLTDPEAAKYVHGIAVHWYLDFLAP
AKATLGETHRLFPNTMLFASEACVGSKFWEQSVRLGSWDRGMQYSHSIIT
NLLYHVVGWTDWNLALNPEGGPNWVRNFVDSPIIVDITKDTFYKQPMFYH
LGHFSKFIPEGSQRVGLVASQKNDLDAVALMHPDGSAVVVVLNRSSKDVP
LTIKDPAVGFLETISPGYSIHTYLWRRQ*

Sequence 24. Amino acid sequence of a glycine-serine linker.

(SEQ ID NO: 24)
GGGGS

Sequence 25. Amino acid sequence of a two repeats of the glycine-serine linker.

(SEQ ID NO: 25)
GGGGSGGGGS

Sequence 26. Amino acid sequence of alternative ICAM-1 targeting peptide.

(SEQ ID NO: 26)
NNQKIVNLKEKVAQLEA

Sequence 27. Amino acid sequence of alternative ICAM-1 targeting peptide.

(SEQ ID NO: 27)
NNQKLVNIKEKVAQIEA

Sequence 28. Amino acid sequence of alternative ICAM-1 targeting peptide.

(SEQ ID NO: 28)
YPASYQR

Sequence 29. Amino acid sequence of alternative ICAM-1 targeting peptide.

(SEQ ID NO: 29)
YQATPLP

Sequence 30. Amino acid sequence of alternative ICAM-1 targeting peptide.

(SEQ ID NO: 30)
GSLLSAA

Sequence 31. Amino acid sequence of alternative ICAM-1 targeting peptide.

(SEQ ID NO: 31)
FSPHSRT

Sequence 32. Amino acid sequence of alternative ICAM-1 targeting peptide.

(SEQ ID NO: 32)
YPFLPTA

Sequence 33. Amino acid sequence of alternative ICAM-1 targeting peptide.

(SEQ ID NO: 33)
GCKLCAQ

Sequence 34. Amino acid sequence of the first protease cleavage site, recognized by cathepsin L or cathepsin B, in the fusion proteins.

(SEQ ID NO: 34)
GFLG

Sequence 35. Amino acid sequence of the second protease cleavage site, the enterokinase cleavage sequence, in the fusion proteins.

(SEQ ID NO: 35)
DDDDK

Sequence 36. Amino acid sequence of the second protease cleavage site, the Tobacco etch virus cleavage sequence, in the fusion proteins.

(SEQ ID NO: 36)
ENLYFQ

Sequence 37. Amino acid sequence of the second protease cleavage site, the Factor Xa cleavage site, in the fusion proteins.

(SEQ ID NO: 37)
IEGR

Sequence 38. Amino acid sequence of the second protease cleavage site, the matrix metalloproteinase 9 (MMP-9) cleavage site, in the fusion proteins.

PXXXX, where X in position 2 and 3 is any residue, position 3 is a hydrophobic residue, and the X in position 5 is S or T (SEQ ID NO:38).

Sequence 39. Amino acid sequence of the second protease cleavage site, the papain cleavage site, in the fusion proteins.

XXXXZRUXXX, where Z is a hydrophobic residue, and U is any residue but V (SEQ ID NO:39)

Sequence 40. Amino acid sequence of the second protease cleavage site, the thrombin cleavage site, in the fusion proteins.

(SEQ ID NO: 40)
LVPRGS

Sequence 41. Amino acid sequence of a secretion signal in the fusion proteins.

(SEQ ID NO: 41)
METDTLLLWVLLLWVPGSTG

Sequence 42. Amino acid sequence of a secretion signal in the fusion proteins.

(SEQ ID NO: 42)
MGWSCIILFLVATATGVHSD

### REFERENCE

He, X., et al. (1999). “Characterization of human acid sphingomyelinase purified from the media of overexpressing Chinese hamster ovary cells.” Biochimica et Biophysica Acta (BBA)-Protein Structure and Molecular Enzymology 1432 (2): 251-264.

The foregoing Examples and Sequences illustrate various embodiments, but do are not intended to limit the disclosure, and those skilled in the art will recognize that various modifications to the Examples and Sequences can be made without departing from the scope of the invention.

What is claimed is:

1. A fusion protein comprising:
i) an adhesion molecule-1 (ICAM-1) targeting segment;
ii) an enzyme segment that is catalytically activated at a lysosomal pH;
iii) a first protease cleavage sequence segment between i) and ii), and optionally, one or more of:
iv) a protein purification tag; and
v) a second protease cleavage sequence.

2. The fusion protein of claim 1, wherein the ICAM-1 targeting segment is repeated at least 2 times in the fusion protein.

3. The fusion protein of claim 2, wherein the repeated ICAM-1 targeting segments are separated from one another by an amino acid linker sequence.

4. The fusion protein of claim 2, wherein the ICAM-1 targeting segment comprises SEQ ID NO:1 (NNQKIVNIKEKVAQIEA (2γ3), NNQKIVNLKEKVAQLEA (SEQ ID NO: 26), NNQKLVNIKEKVAQIEA (SEQ ID NO: 27), YPASYQR (SEQ ID NO: 28), YQATPLP (SEQ ID NO: 29), GSLLSAA (SEQ ID NO: 30), FSPHSRT (SEQ ID NO: 31), YPFLPTA (SEQ ID NO: 32), GCKLCAQ (SEQ ID NO: 33), and a combination thereof.

5. The fusion protein of claim 4, wherein the ICAM-1 targeting segment is repeated 2-10 times.

6. The fusion protein of claim 5, wherein the first protease cleavage sequence comprises a cathepsin L or cathepsin B protease cleavage site.

7. The fusion protein of claim 6, wherein the first protease cleavage sequence comprises a cathepsin B cleavage site.

8. The fusion protein of claim 7, wherein the fusion protein is within a eukaryotic cell.

9. A method comprising introducing the fusion protein of claim 1 into a eukaryotic cell.

10. The method of claim 9, wherein the ICAM-1 targeting segment is repeated at least 2 times in the fusion protein.

11. The method of claim 10, wherein the repeated ICAM-1 targeting segments are separated from one another by an amino acid linker sequence.

12. The method of claim 10, wherein the ICAM-1 targeting segment comprises an amino acid sequence selected from the group of amino acid sequences consisting of: SEQ ID NO:1 (NNQKIVNIKEKVAQIEA (2γ3), NNQKIVNLKEKVAQLEA (SEQ ID NO: 26), NNQKLVNIKEKVAQIEA (SEQ ID NO: 27), YPASYQR (SEQ ID NO: 28), YQATPLP (SEQ ID NO: 29), GSLLSAA (SEQ ID NO: 30), FSPHSRT (SEQ ID NO: 31), YPFLPTA (SEQ ID NO: 32), GCKLCAQ (SEQ ID NO: 33), and a combination thereof.

13. The method of claim 12, wherein the ICAM-1 targeting segment is repeated 2-10 times.

14. The method of claim 13, wherein the first protease cleavage sequence comprises a cathepsin L cleavage site or a cathepsin B protease cleavage site.

15. The method of claim 14, wherein the fusion protein is cleaved at the first protease cleavage sequence such that the enzyme segment is separated from the fusion protein within a lysosome of the eukaryotic cell, and wherein the protease cleavage site comprises a cathepsin B protease cleavage site.

16. The method of claim 15, wherein the eukaryotic cell is present in an individual who has been diagnosed with a lysosomal storage disease.

17. An expression vector encoding the fusion protein of claim 1.

18. The expression vector of claim 17, wherein the ICAM-1 targeting segment is repeated at least 2 times in the fusion protein.

19. The expression vector of claim 11, wherein the ICAM-1 targeting segment comprises an amino acid sequence selected from the group of amino acid sequences consisting of: SEQ ID NO:1 (NNQKIVNIKEKVAQIEA (2γ3), NNQKIVNLKEKVAQLEA (SEQ ID NO: 26), NNQKLVNIKEKVAQIEA (SEQ ID NO: 27), YPASYQR (SEQ ID NO: 28), YQATPLP (SEQ ID NO: 29), GSLLSAA (SEQ ID NO: 30), FSPHSRT (SEQ ID NO: 31), YPFLPTA (SEQ ID NO: 32), GCKLCAQ (SEQ ID NO: 33).

20. A cell comprising the expression vector of claim 17.


SEQUENCE LISTING (42 sequences)

SEQ ID NO: 1 - protein, 17 aa, Homo sapiens
NNQKIVNIKEKVAQIEA

SEQ ID NO: 2 - DNA, 1902 bp, synthetic construct
  misc_feature 1..1902: note = cDNA sequence of the expression cassette for human acid sphingomyelinase (ASM) with one copy of the 2gamma3 ICAM-1-targeting peptide at the amino terminus
  source 1..1902: mol_type = other DNA
atggagaccgacacactgctcctgtgggtcctgctcctctgggtgccaggaagtacaggagatcaccatcaccatcaccacgacgacgacgacaagaataaccaaaagattgtgaatatcaaagagaaagtggctcagattgaggctggaggcggaggaagcggcggcggaggaagcggatttctgggacaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 3 - DNA, 2226 bp, synthetic construct
  misc_feature 1..2226: note = cDNA sequence of the expression cassette for human ASM with five tandem-repeats of the 2?3 ICAM-1-targeting peptide at the amino terminus.
  source 1..2226: mol_type = other DNA
atggagaccgacacactgctcctgtgggtcctgctcctctgggtgccaggaagtacaggagatcaccatcaccatcaccacgacgacgacgacaagaataaccaaaagattgtgaatatcaaagagaaagtggctcagattgaggctggaggcggaggaagcggcggcggaggaagcaataatcagaaaatcgtcaacattaaggaaaaggtcgcccagattgaagcaggaggcggcggcagcggcggaggcggaagcaataatcagaagattgttaacatcaaagaaaaggtggcccaaattgaagcaggaggaggaggatctggaggcggaggcagcaataaccagaagatcgtcaacatcaaggaaaaggtggctcagatcgaggcaggaggcggaggaagcggagggggcggctctaacaaccagaaaatcgtgaacatcaaagagaaagtggctcagatcgaagccggcggaggaggatccggaggaggaggaagcggatttctgggacaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 4 - DNA, 2631 bp, synthetic construct
  misc_feature 1..2631: note = cDNA sequence of the expression cassette for human ASM with ten tandem-repeats of the 2gamma3 ICAM-1-targeting peptide at the amino terminus
  source 1..2631: mol_type = other DNA
atggagaccgacacactgctcctgtgggtcctgctcctctgggtgccaggaagtacaggagatcaccatcaccatcaccacgacgacgacgacaagaataaccaaaagattgtgaatatcaaagagaaagtggctcagattgaggctggaggcggaggaagcggcggcggaggaagcaataatcagaaaatcgtcaacattaaggaaaaggtcgcccagattgaagcaggaggcggcggcagcggcggaggcggaagcaataatcagaagattgttaacatcaaagaaaaggtggcccaaattgaagcaggaggaggaggatctggaggcggaggcagcaataaccagaagatcgtcaacatcaaggaaaaggtggctcagatcgaggcaggaggcggaggaagcggagggggcggctctaacaaccagaaaatcgtgaacatcaaagagaaagtggctcagatcgaagccggcggaggaggatccggaggaggaggaagcaataaccaaaagattgtgaatatcaaagagaaagtggctcagattgaggctggaggcggaggaagcggcggcggaggaagcaataatcagaaaatcgtcaacattaaggaaaaggtcgcccagattgaagcaggaggcggcggcagcggcggaggcggaagcaataatcagaagattgttaacatcaaagaaaaggtggcccaaattgaagcaggaggaggaggatctggaggcggaggcagcaataaccagaagatcgtcaacatcaaggaaaaggtggctcagatcgaggcaggaggcggaggaagcggagggggcggctctaacaaccagaaaatcgtgaacatcaaagagaaagtggctcagatcgaagccggcggaggaggatccggaggaggaggaagcggatttctgggacaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 5 - DNA, 2226 bp, synthetic construct
  misc_feature 1..2226: note = cDNA sequence of the expression cassette for human ASM with five tandem-repeats of the 2gamma3 ICAM-1-targeting peptide at the carboxyl terminus
  source 1..2226: mol_type = other DNA
atggagaccgacacactgctcctgtgggtcctgctcctctgggtgccaggaagtacaggagatcaccatcaccatcaccacgacgacgacgacaagcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgcggatttctgggaggcggaggaggatccggaggaggaggaagcaataaccaaaagattgtgaatatcaaagagaaagtggctcagattgaggctggaggcggaggaagcggcggcggaggaagcaataatcagaaaatcgtcaacattaaggaaaaggtcgcccagattgaagcaggaggcggcggcagcggcggaggcggaagcaataatcagaagattgttaacatcaaagaaaaggtggcccaaattgaagcaggaggaggaggatctggaggcggaggcagcaataaccagaagatcgtcaacatcaaggaaaaggtggctcagatcgaggcaggaggcggaggaagcggagggggcggctctaacaaccagaaaatcgtgaacatcaaagagaaagtggctcagatcgaagcctag

SEQ ID NO: 6 - DNA, 1809 bp, synthetic construct
  misc_feature 1..1809: note = cDNA sequence of the expression cassette for human ASM control
  source 1..1809: mol_type = other DNA
atggagaccgacacactgctcctgtgggtcctgctcctctgggtgccaggaagtacaggagatcaccatcaccatcaccacgacgacgacgacaagcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 7 - DNA, 1383 bp, synthetic construct
  misc_feature 1..1383: note = cDNA sequence of the expression cassette for human alpha galactosidase (?Gal) with one copy of the 2gamma3 ICAM-1-targeting peptide at the amino terminus
  source 1..1383: mol_type = other DNA
atgggctggtcctgcatcattctgtttctggtggctaccgccaccggcgtgcactctgatcaccaccaccatcaccacgacgatgacgacaagaacaaccagaagatcgtcaacatcaaagagaaggtcgcccagatcgaggctggaggcggaggatctggtggtggcggatctggattccttggcctggacaacggcctggctagaacccctaccatgggatggctgcactgggagagattcatgtgcaacctggactgccaagaggaacccgactcctgcatctccgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcatcagacagctggccaactacgtgcactccaagggcctgaagctgggcatctatgccgacgtgggcaacaagacctgtgccggctttcctggctccttcggctactacgatatcgacgcccagaccttcgctgactggggagtcgatctgctgaagttcgacggctgctactgcgactccctggaaaatctggccgacggctacaagcacatgtctctggccctgaaccggaccggcagatccatcgtgtatagctgcgagtggcccctgtacatgtggcccttccagaagcctaactacaccgagatcagacagtactgcaaccactggcggaacttcgccgacatcgacgatagctggaagtccatcaagtctatcctggactggacctccttcaatcaagagcggatcgtggatgtggctggccctggcggatggaacgatcctgatatgctggtcatcggcaacttcggcctgtcctggaaccagcaagtgacccagatggccctgtgggccattatggccgctcctctgttcatgtccaacgacctgagacacatcagccctcaggccaaggctctgctgcaggacaaggatgtgatcgctatcaaccaggatcctctgggcaagcagggctaccagttgagacagggcgacaactttgaagtgtgggaaagacccctgtccggcctggcatgggctgtcgccatgatcaacagacaagagatcggcggaccccggtcctacacaatcgctgttgcttctctcggcaaaggcgtggcctgcaatcctgcctgtttcatcacacagctgctgcccgtgaagagaaagctgggcttttacgagtggacctctcggctgcggtcccacatcaatcctaccggaacagtgctgctgcagctggaaaacaccatgcagatgtccctgaaggacctgctgtga

SEQ ID NO: 8 - DNA, 1707 bp, synthetic construct
  misc_feature 1..1707: note = cDNA sequence of the expression cassette for human ?Gal with five tandem-repeats of the 2?3 ICAM-1-targeting peptide at the carboxyl terminus
  source 1..1707: mol_type = other DNA
atgggctggtcctgcatcattctgtttctggtggctaccgccaccggcgtgcactctgatcaccaccaccatcaccacgacgatgacgacaagctggacaacggcctggctagaacccctaccatgggatggctgcactgggagagattcatgtgcaacctggactgccaagaggaacccgactcctgcatctccgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcatcagacagctggccaactacgtgcactccaagggcctgaagctgggcatctatgccgacgtgggcaacaagacctgtgccggctttcctggctccttcggctactacgatatcgacgcccagaccttcgctgactggggagtcgatctgctgaagttcgacggctgctactgcgactccctggaaaatctggccgacggctacaagcacatgtctctggccctgaaccggaccggcagatccatcgtgtatagctgcgagtggcccctgtacatgtggcccttccagaagcctaactacaccgagatcagacagtactgcaaccactggcggaacttcgccgacatcgacgatagctggaagtccatcaagtctatcctggactggacctccttcaatcaagagcggatcgtggatgtggctggccctggcggatggaacgatcctgatatgctggtcatcggcaacttcggcctgtcctggaaccagcaagtgacccagatggccctgtgggccattatggccgctcctctgttcatgtccaacgacctgagacacatcagccctcaggccaaggctctgctgcaggacaaggatgtgatcgctatcaaccaggatcctctgggcaagcagggctaccagttgagacagggcgacaactttgaagtgtgggaaagacccctgtccggcctggcatgggctgtcgccatgatcaacagacaagagatcggcggaccccggtcctacacaatcgctgttgcttctctcggcaaaggcgtggcctgcaatcctgcctgtttcatcacacagctgctgcccgtgaagagaaagctgggcttttacgagtggacctctcggctgcggtcccacatcaatcctaccggaacagtgctgctgcagctggaaaacaccatgcagatgtccctgaaggacctgctgggattccttggcggaggcggaggatctggtggtggcggatctaacaaccagaagatcgtcaacatcaaagagaaggtcgcccagatcgaggctggcggcggtggatcaggtggcggaggaagcaacaatcagaaaattgtgaatatcaaagaaaaagtggctcagattgaagcaggcggcggaggtagcggaggtggtggctctaacaatcaaaaaatcgttaacatcaaagagaaagttgctcaaatcgaagccggcggtggtggttctggcggtggtggtagtaacaatcaaaagatcgtcaatatcaaagaaaaggtggcacaaatcgaggcaggcggaggcggctctggcggcggaggatcaaacaatcagaagatcgttaacatcaaagaaaaagtggcccaaattgaggcctga

SEQ ID NO: 9 - DNA, 1290 bp, synthetic construct
  misc_feature 1..1290: note = cDNA sequence of the expression cassette for human ?Gal control
  source 1..1290: mol_type = other DNA
atgggctggtcctgcatcattctgtttctggtggctaccgccaccggcgtgcactctgatcaccaccaccatcaccacgacgatgacgacaagctggacaacggcctggctagaacccctaccatgggatggctgcactgggagagattcatgtgcaacctggactgccaagaggaacccgactcctgcatctccgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcatcagacagctggccaactacgtgcactccaagggcctgaagctgggcatctatgccgacgtgggcaacaagacctgtgccggctttcctggctccttcggctactacgatatcgacgcccagaccttcgctgactggggagtcgatctgctgaagttcgacggctgctactgcgactccctggaaaatctggccgacggctacaagcacatgtctctggccctgaaccggaccggcagatccatcgtgtatagctgcgagtggcccctgtacatgtggcccttccagaagcctaactacaccgagatcagacagtactgcaaccactggcggaacttcgccgacatcgacgatagctggaagtccatcaagtctatcctggactggacctccttcaatcaagagcggatcgtggatgtggctggccctggcggatggaacgatcctgatatgctggtcatcggcaacttcggcctgtcctggaaccagcaagtgacccagatggccctgtgggccattatggccgctcctctgttcatgtccaacgacctgagacacatcagccctcaggccaaggctctgctgcaggacaaggatgtgatcgctatcaaccaggatcctctgggcaagcagggctaccagttgagacagggcgacaactttgaagtgtgggaaagacccctgtccggcctggcatgggctgtcgccatgatcaacagacaagagatcggcggaccccggtcctacacaatcgctgttgcttctctcggcaaaggcgtggcctgcaatcctgcctgtttcatcacacagctgctgcccgtgaagagaaagctgggcttttacgagtggacctctcggctgcggtcccacatcaatcctaccggaacagtgctgctgcagctggaaaacaccatgcagatgtccctgaaggacctgctgtga

SEQ ID NO: 10 - DNA, 1680 bp, synthetic construct
  misc_feature 1..1680: note = cDNA sequence of the expression cassette for human glucocerebrosidase (GCase) with one copy of the 2gamma ICAM-1-targeting peptide at the amino terminus
  source 1..1680: mol_type = other DNA
atgggctggtcctgcatcattctgtttctggtggctaccgccaccggcgtgcactctgatcaccaccaccatcaccacgacgatgacgacaagaacaaccagaagatcgtcaacatcaaagagaaggtcgcccagatcgaggctggcggcggaggatctggcggaggcggatctggatttttgggagccagaccttgcatccccaagtccttcggctactcctctgtcgtgtgcgtgtgcaacgccacctactgcgacagcttcgaccctcctacctttcctgctctgggcacattctccagatacgagtccaccagatccggcagacggatggaactgagcatgggccctatccaggctaaccataccggcacaggactgctgctgacactgcagcccgagcagaaattccagaaagtgaaaggcttcggcggagccatgaccgatgccgccgctctgaatattctggctctgagccctcctgctcagaacctgctgctcaagtcctacttctccgaggaaggcatcggctacaacatcatccgggtgccaatggcctcctgcgacttctctatccggacctacacctacgctgacacccctgacgatttccagctgcacaacttcagcctgcctgaagaggacaccaagctgaagatccctctgatccacagagccctgcagctggctcagaggcctgtttctctgctggcctctccttggacctctccaacctggctgaaaacaaatggcgccgtgaacggcaagggctccctgaaaggacaacccggcgatatctaccaccagacctgggccagatacttcgtgaagttcctggacgcctacgccgagcacaagctgcagttttgggctgtgaccgccgagaacgagccttctgctggactgctgtctggctaccctttccagtgcctgggctttacccctgagcaccagagagactttatcgccagagatctgggccccacactggccaattctacccaccataatgtgcggctgctgatgctggacgaccagagactgctgttgccccactgggctaaagtggtgctgaccgatcctgaggccgccaaatacgtgcacggaatcgccgtgcactggtatctggactttctggcccctgccaaggctaccctgggcgagacacatagactgttccccaacaccatgctgttcgcctctgaggcctgtgtgggctccaagttctgggagcagtctgtgcgactcggctcttgggatagaggcatgcagtactcccactccatcatcaccaacctgctgtaccacgtcgtcggctggaccgattggaacctggcactgaatcctgaaggcggccctaactgggtccgaaacttcgtggactcccctatcatcgtggacatcaccaaggacaccttctacaagcagcccatgttctaccatctgggccacttcagcaagttcatccccgagggctctcagagagtcggcctggttgcctctcagaagaacgacctggacgctgtggctctgatgcaccctgatggatctgctgtggtggtcgtgctgaaccggtcctccaaagatgtgcccctgaccatcaaggatcccgccgtgggattcctggaaaccatctctcctggctactccatccacacctacctgtggcgtagacagtga

SEQ ID NO: 11 - DNA, 2004 bp, synthetic construct
  misc_feature 1..2004: note = cDNA sequence of the expression cassette for human GCase with five tandem-repeats of the 2?gamma ICAM-1-targeting peptide at the amino terminus
  source 1..2004: mol_type = other DNA
atgggctggtcctgcatcattctgtttctggtggctaccgccaccggcgtgcactctgatcaccaccaccatcaccacgacgatgacgacaagaacaaccagaagatcgtcaacatcaaagagaaggtcgcccagatcgaggctggcggcggaggatctggcggaggcggatctaacaatcagaaaattgtgaatatcaaagaaaaagtggctcagattgaagccggcggtggtggtagcggtggcggaggaagtaacaatcaaaagatcgtgaacatcaaagaaaaagttgcacaaatcgaggcaggcggtggcggcagcggaggtggtggatccaacaaccagaaaatcgtgaacatcaaagaaaaggtggcccaaatcgaagccggcggaggcggttcaggcggcggaggttcaaacaatcagaagatcgttaatatcaaagaaaaggttgcccagattgaggcaggcggaggtggaagcggcggaggcggctctggatttttgggagccagaccttgcatccccaagtccttcggctactcctctgtcgtgtgcgtgtgcaacgccacctactgcgacagcttcgaccctcctacctttcctgctctgggcacattctccagatacgagtccaccagatccggcagacggatggaactgagcatgggccctatccaggctaaccataccggcacaggactgctgctgacactgcagcccgagcagaaattccagaaagtgaaaggcttcggcggagccatgaccgatgccgccgctctgaatattctggctctgagccctcctgctcagaacctgctgctcaagtcctacttctccgaggaaggcatcggctacaacatcatccgggtgccaatggcctcctgcgacttctctatccggacctacacctacgctgacacccctgacgatttccagctgcacaacttcagcctgcctgaagaggacaccaagctgaagatccctctgatccacagagccctgcagctggctcagaggcctgtttctctgctggcctctccttggacctctccaacctggctgaaaacaaatggcgccgtgaacggcaagggctccctgaaaggacaacccggcgatatctaccaccagacctgggccagatacttcgtgaagttcctggacgcctacgccgagcacaagctgcagttttgggctgtgaccgccgagaacgagccttctgctggactgctgtctggctaccctttccagtgcctgggctttacccctgagcaccagagagactttatcgccagagatctgggccccacactggccaattctacccaccataatgtgcggctgctgatgctggacgaccagagactgctgttgccccactgggctaaagtggtgctgaccgatcctgaggccgccaaatacgtgcacggaatcgccgtgcactggtatctggactttctggcccctgccaaggctaccctgggcgagacacatagactgttccccaacaccatgctgttcgcctctgaggcctgtgtgggctccaagttctgggagcagtctgtgcgactcggctcttgggatagaggcatgcagtactcccactccatcatcaccaacctgctgtaccacgtcgtcggctggaccgattggaacctggcactgaatcctgaaggcggccctaactgggtccgaaacttcgtggactcccctatcatcgtggacatcaccaaggacaccttctacaagcagcccatgttctaccatctgggccacttcagcaagttcatccccgagggctctcagagagtcggcctggttgcctctcagaagaacgacctggacgctgtggctctgatgcaccctgatggatctgctgtggtggtcgtgctgaaccggtcctccaaagatgtgcccctgaccatcaaggatcccgccgtgggattcctggaaaccatctctcctggctactccatccacacctacctgtggcgtagacagtga

SEQ ID NO: 12 - DNA, 1290 bp, synthetic construct; identical to SEQ ID NO: 9
  misc_feature 1..1290: note = cDNA sequence of the expression cassette for human GCase control
  source 1..1290: mol_type = other DNA

SEQ ID NO: 13 - protein, 633 aa, synthetic construct
  REGION 1..633: note = Amino acid sequence of the expression cassette for human acid sphingomyelinase (ASM) with one copy of the 2gamma3 ICAM-1-targeting peptide at the amino terminus
METDTLLLWVLLLWVPGSTGDHHHHHHDDDDKNNQKIVNIKEKVAQIEAGGGGSGGGGSGFLGHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 14 - protein, 741 aa, synthetic construct
  REGION 1..741: note = Amino acid sequence of the expression cassette for human ASM with five tandem-repeats of the 2?gamma ICAM-1-targeting peptide at the amino terminus
METDTLLLWVLLLWVPGSTGDHHHHHHDDDDKNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSGFLGHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 15 - protein, 876 aa, synthetic construct
  REGION 1..876: note = Amino acid sequence of the expression cassette for human ASM with ten tandem-repeats of the 2gamma3 ICAM-1-targeting peptide at the amino terminus
METDTLLLWVLLLWVPGSTGDHHHHHHDDDDKNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSGFLGHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 16 - protein, 741 aa, synthetic construct
  REGION 1..741: note = Amino acid sequence of the expression cassette for human ASM with five tandem-repeats of the 2?gamma ICAM-1-targeting peptide at the carboxyl terminus
METDTLLLWVLLLWVPGSTGDHHHHHHDDDDKHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFCGFLGGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEA

SEQ ID NO: 17 - protein, 602 aa, synthetic construct
  REGION 1..602: note = Amino acid sequence of the expression cassette for human ASM control
METDTLLLWVLLLWVPGSTGDHHHHHHDDDDKHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 18 - protein, 460 aa, synthetic construct
  REGION 1..460: note = Amino acid sequence of the expression cassette for human alpha galactosidase with one copy of the 2gamma3 ICAM-1-targeting peptide at the amino terminus
MGWSCIILFLVATATGVHSDHHHHHHDDDDKNNQKIVNIKEKVAQIEAGGGGSGGGGSGFLGLDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 19 - protein, 568 aa, synthetic construct
  REGION 1..568: note = Amino acid sequence of the expression cassette for human alphaGal with five tandem-repeats of the 2beta3 ICAM-1-targeting peptide at the carboxyl terminus
MGWSCIILFLVATATGVHSDHHHHHHDDDDKLDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLLGFLGGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEA

SEQ ID NO: 20 - protein, 429 aa, synthetic construct
  REGION 1..429: note = Amino acid sequence of the expression cassette for human alphaGal control
MGWSCIILFLVATATGVHSDHHHHHHDDDDKLDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 21 - protein, 559 aa, synthetic construct
  REGION 1..559: note = Amino acid sequence of the expression cassette for human glucocerebrosidase (GCase) with one copy of the 2gamma3 ICAM-1-targeting peptide at the amino terminus
MGWSCIILFLVATATGVHSDHHHHHHDDDDKNNQKIVNIKEKVAQIEAGGGGSGGGGSGFLGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 22 - protein, 667 aa, synthetic construct
  REGION 1..667: note = Amino acid sequence of the expression cassette for human GCase with five tandem-repeats of the 2gamma3 ICAM-1-targeting peptide at the amino terminus
MGWSCIILFLVATATGVHSDHHHHHHDDDDKNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSNNQKIVNIKEKVAQIEAGGGGSGGGGSGFLGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 23 - protein, 528 aa, synthetic construct
  REGION 1..528: note = Amino acid sequence of the expression cassette for human GCase control
MGWSCIILFLVATATGVHSDHHHHHHDDDDKARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 24 - protein, 5 aa, synthetic construct
  REGION 1..5: note = Amino acid sequence of a glycine-serine linker
GGGGS

SEQ ID NO: 25 - protein, 10 aa, synthetic construct
  REGION 1..10: note = Amino acid sequence of a two repeats of the glycine-serine linker
GGGGSGGGGS

SEQ ID NO: 26 - protein, 17 aa, synthetic construct
  REGION 1..17: note = Amino acid sequence of alternative ICAM-1 targeting peptide
NNQKIVNLKEKVAQLEA

SEQ ID NO: 27 - protein, 17 aa, synthetic construct
  REGION 1..17: note = Amino acid sequence of alternative ICAM-1 targeting peptide
NNQKLVNIKEKVAQIEA

SEQ ID NO: 28 - protein, 7 aa, synthetic construct
  REGION 1..7: note = Amino acid sequence of alternative ICAM-1 targeting peptide
YPASYQR

SEQ ID NO: 29 - protein, 7 aa, synthetic construct
  REGION 1..7: note = Amino acid sequence of alternative ICAM-1 targeting peptide
YQATPLP

SEQ ID NO: 30 - protein, 7 aa, synthetic construct
  REGION 1..7: note = Amino acid sequence of alternative ICAM-1 targeting peptide
GSLLSAA

SEQ ID NO: 31 - protein, 7 aa, synthetic construct
  REGION 1..7: note = Amino acid sequence of alternative ICAM-1 targeting peptide
FSPHSRT

SEQ ID NO: 32 - protein, 7 aa, synthetic construct
  REGION 1..7: note = Amino acid sequence of alternative ICAM-1 targeting peptide
YPFLPTA

SEQ ID NO: 33 - protein, 7 aa, synthetic construct
  REGION 1..7: note = Amino acid sequence of alternative ICAM-1 targeting peptide
GCKLCAQ

SEQ ID NO: 34 - protein, 4 aa, synthetic construct
  REGION 1..4: note = Amino acid sequence of the first protease cleavage site, recognized by cathepsin L or cathepsin B, in the fusion proteins
GFLG

SEQ ID NO: 35 - protein, 5 aa, synthetic construct
  REGION 1..5: note = Amino acid sequence of the second protease cleavage site, the enterokinase cleavage sequence, in the fusion proteins
DDDDK

SEQ ID NO: 36 - protein, 6 aa, synthetic construct
  REGION 1..6: note = Amino acid sequence of the second protease cleavage site, the Tobacco etch virus cleavage sequence, in the fusion proteins
ENLYFQ

SEQ ID NO: 37 - protein, 4 aa, synthetic construct
  REGION 1..4: note = Amino acid sequence of the second protease cleavage site, the Factor Xa cleavage site, in the fusion proteins
IEGR

SEQ ID NO: 38-39 - skipped (2 entries)

SEQ ID NO: 40 - protein, 6 aa, synthetic construct
  REGION 1..6: note = Amino acid sequence of the second protease cleavage site, the thrombin cleavage site, in the fusion proteins
LVPRGS

SEQ ID NO: 41 - protein, 20 aa, synthetic construct
  REGION 1..20: note = Amino acid sequence of a secretion signal in the fusion protein
METDTLLLWVLLLWVPGSTG

SEQ ID NO: 42 - protein, 20 aa, synthetic construct
  REGION 1..20: note = Amino acid sequence of a secretion signal in the fusion proteins
MGWSCIILFLVATATGVHSD